(19) United States
(12) Patent Application Publication
(10) Pub. No.: US 2025/0332433 A1
(43) Pub. Date: Oct. 30, 2025

(54) Variable Human-Safe Electromagnetic Radiation Therapy Systems and Methods

(71) Applicant: Light Matters Solutions, Inc., Merritt Island, FL (US)

(72) Inventors: Jesslynn Stull, Orlando, FL (US); Rafael Andrade, Orlando, FL (US); Pedro Medelius, Merritt Island, FL (US)

(73) Assignee: Light Matters Solutions, Inc., Merritt Island, FL (US)

(21) Appl. No.: 18/116,299

(22) Filed: Mar. 1, 2023

(51) Int. Cl. A61N 5/06

(52) U.S. Cl. CPC A61N 5/06; A61N 2005/0626; A61N 2005/0659; A61N 2005/0661; A61N 2005/0662

(57) ABSTRACT

A system may include a therapy device including one or more therapy modules configured to couple to a patient. Each therapy module includes an array of radiation-emitting components, a waveform generator, and a processor coupled to the waveform generator and to the array. Each emitting component is configured to emit electromagnetic radiation in response to an electrical signal. The waveform generator is coupled to the array of radiation-emitting components and configured to generate one or more time-varying waveforms to drive the radiation-emitting components. The processor may be configured to select a protocol of a plurality of protocols. Each protocol may define one or more parameters including one or more of a waveform shape, an amplitude, a frequency, a timing parameter, and a duration parameter. The processor may be configured to send a signal to the waveform generator to generate the one or more time-varying waveforms according to the selected protocol.

### FIELD

The present disclosure is generally related to variable human-safe radiation therapy systems and methods, and more particularly, to systems, methods, and devices configured to provide a therapeutic effect using emitted radiation, such as electromagnetic signals including one or more of magnetic fields, electrical fields, or time-varying light emissions within a range of the light spectrum from infrared light to visible light to ultraviolet light.

### BACKGROUND

Visible and near infrared wavelength light is known to have many therapeutic benefits. For example, wavelengths of 680, 730 and/or 880 nanometers have been shown to increase cell growth and speed wound healing in patients. Conventionally, light-therapy devices are available that combine infrared light and a visible red light for treating sicknesses, illnesses, and injuries, such as strained muscles, myalgia, joint pains, headaches, dermal inflammation, wounds, or any combination thereof.

### SUMMARY

Embodiments of systems, methods, and devices are described below that may be configured to treat injuries and mitigate pain. Such injuries may include muscle pains (including pain associated with ligaments, tendons, and soft tissues), joint pains, skin inflammation, wounds, and so on. The system may include a plurality of radiation-emitting components configured to emit one or more of electromagnetic radiation at selected wavelengths (including visible light, ultraviolet light, infrared light, photons, magnetic fields, electrical fields, other electromagnetic radiation, or any combination thereof).

The radiation-emitting components may be arranged in an array and embedded in a surface of a device that may contact the skin directly or indirectly via a transparent coating (such as a medicinal coating or a transparent bandage). The system may include a microcontroller unit configured to independently control one or more of the radiation-emitting components to emit electromagnetic radiation at a selected wavelength and at selected modulations, such as amplitude, timing, and duration. In some implementations, the microcontroller may be configured to control the radiation-emitting components according to a predetermined pattern. The system may include one or more sensors coupled to the microcontroller and configured to generate signals indicative of one or more parameters associated with a patient and to communicate the generated signals to the microcontroller.

In some implementations, the one or more sensors may include an optical sensor configured to generate electrical signals indicative of coherent structures in living tissue. In particular, the optical sensor may be configured to generate electrical signals indicative of birefringence of the tissue, which may be indicative of structured water molecules aligned to the coherent structures of the tissue. Sensor signals indicative of a high level of birefringence may be indicative of effectiveness and extend of the patient's response to the selected treatment.

In some implementations, one or more of the sensors may be integrated within a therapy device or therapy module. In addition to or in lieu of the sensors integrated in the therapy device, one or more other sensors may be applied to a patient or may be incorporated into wearable devices. Each of the sensors may communicate signals or data determined from the signals to the microcontroller.

The microcontroller may be configured to selectively control the radiation-emitting components in response to one or more of the predetermined patterns based on the signals received from the sensors. In some implementations, the microcontroller may be configured to utilize artificial intelligence (AI) and machine learning (ML) to determine self-configuring patterns based on the sensor data. In other implementations, the microcontroller may communicate data to an analytics system, which may be configured to produce and provide self-configuring patterns by using one or more of an artificial intelligence system or machine learning to process the sensor data. The analytics system may communicate the self-configuring patterns to the microcontroller, which may apply them for delivering electromagnetic or photonic radiation to the patient.

In some implementations, the microcontroller unit (MCU) may control the plurality of radiation-emitting components using electrical signals to selectively direct electromagnetic radiation toward the patient. The MCU may modulate the electrical signals of one or more selected wavelengths, one or more amplitudes, and one or more durations, and at selected relative timing to selected ones of the radiation-emitting components to achieve a physiological effect. In some implementations, the MCU may be configured to control each emitting component independently. Additionally, the MCU may receive signals from one or more sensors and may selectively adjust one or more of the wavelengths or the corresponding modulation (the amplitudes, the durations, or the timings of emitted radiation from the various radiation-emitting components) to provide the selected physiological effect. In some implementations, the device may be configured to fit a treatment area of the user's body and optionally may be releasably coupled to the user or otherwise applied to the treatment area.

In some implementations, a system may include one or more therapy modules configured to couple to a patient. Each therapy module may include a communicatons interface, an array of radiation-emitting components, a waveform generator, and a processor coupled to the communications interface, the waveform generator, and the array. Each emitting component may be configured to emit electromagnetic radiation in response to an electrical signal received from the waveform generator, which may be coupled to the array of radiation-emitting components and which may be configured to generate one or more time-varying waveforms to drive the radiation-emitting components. The processor may be configured to select a protocol of a plurality of protocols automatically or in response to a signal received at the communications interface. Each protocol may define one or more parameters including one or more of a waveform shape, an amplitude, a wavelength, a timing parameter, and a duration parameter. The processor may be configured to send a signal to the waveform generator to generate the one or more time-varying waveforms according to the selected protocol, which may drive radiation emitting elements of the array of radiation-emitting elements to emit electromagnetic radiation having selected wavelengths toward the patient.

In other implementations, a system may include a control device including an input interface to receive input data and a communications interface configured to send signals indicative of the received input data to one or more therapy modules, each of which may be configured to couple to a patient. Each therapy module may include a communications interface configured to determine a communications link between the communications interface and one or more of the control device or a second therapy module. Each therapy module may include a processor, an array of radiation-emitting components configured to emit electromagnetic radiation toward a treatment area of the patient and a waveform generator coupled to the array and to the processor and configured to generate one or more time-varying waveforms in response to a control signal. The processor may be configured to receive data related to the input data from the communications interface, determine a selected protocol from a plurality of protocols based on the received data, and send the control signal to the waveform generator based on the selected protocol to treat one or more treatment areas on the patient according to the selected protocol.

In still other implementations, a system may include a plurality of therapy modules configured to couple to one or more treatment areas on a patient. Each therapy module may include a communications interface configured to selectively establish a communications link to one or more of a control device or one or more other therapy modules to cover one or more treatment areas having a selected size and shape. Each therapy module may include an array of radiation-emitting components configured to emit electromagnetic radiation toward one of the one or more treatment areas. Each therapy module may include a waveform generator coupled to the array and configured to generate one or more time-varying waveforms to drive the radiation-emitting components individually, in subsets, or in total. Each therapy module may include a processor configured to select a protocol from a plurality of protocols and control the waveform generator to generate the one or more time-varying waveforms based on the selected protocol.

### BRIEF DESCRIPTION OF THE DRAWINGS

The detailed description is set forth with reference to the accompanying figures. In the figures, the left most digit(s) of a reference number identifies the figure in which the reference number first appears. The use of the same reference numbers in different figures indicates similar or identical items or features.

FIG. 1 depicts a block diagram of a system including a therapy system comprised of a plurality of therapy modules and a control device, in accordance with certain embodiments of the present disclosure.

FIG. 2 depicts a block diagram of an implementation of the control device of FIG. 1, in accordance with certain embodiments of the present disclosure.

FIG. 3 depicts a block diagram of a system including an implementation of an analytics system configured to communicate with one or more of computing devices or a control system, through a communications network, in accordance with certain embodiments of the present disclosure.

FIG. 4A depicts a diagram of a modular therapy system comprised of a plurality of right-triangular shaped therapy modules, in accordance with certain embodiments of the present disclosure.

FIG. 4B depicts a diagram of a modular therapy system comprised of a plurality of equilateral-triangular shaped therapy modules, in accordance with certain embodiments of the present disclosure.

FIG. 4C depicts a diagram of a modular therapy system comprised of a plurality of hexagon-shaped therapy modules, in accordance with certain embodiments of the present disclosure.

FIG. 5 depicts diagrams of various shapes of therapy modules, in accordance with certain embodiments of the present disclosure.

FIG. 6A depicts a diagram of a therapy module, in accordance with certain embodiments of the present disclosure.

FIG. 6B depicts a cross-sectional view of the therapy module of FIG. 6A taken along line BC-BC in FIG. 6A, in accordance with certain embodiments of the present disclosure.

FIG. 6C depicts a cross-sectional view of the therapy module of FIG. 6A taken along line BC-BC in FIG. 6A, in accordance with certain embodiments of the present disclosure.

FIG. 7 depicts a graph of electrical signals configured to drive selected radiation-emitting components of a modular therapy device and having time-varying signal shapes, signal amplitudes, signal wavelengths, and relative timing of emitted electromagnetic radiation, in accordance with certain embodiments of the present disclosure.

FIG. 8 depicts wearable devices that may include integrated therapy modules configurable to provide a selected therapeutic effect, in accordance with certain embodiments of the present disclosure.

FIG. 9 depicts a plurality of therapy modules applied directly to a patient's skin to form a distributed therapy device configurable to provide a selected therapeutic effect, in accordance with certain embodiments of the present disclosure.

FIG. 10 depicts a flow diagram of a method of monitoring one or more parameters associated with a patient, in accordance with certain embodiments of the present disclosure.

FIG. 11 depicts a flow diagram of a method of selectively adjusting one or more parameters of a selected treatment mode, in accordance with certain embodiments of the present disclosure.

FIG. 12 depicts a flow diagram of a method of selectively adjusting one or more parameters of signals provided to radiation-emitting components of the therapy module, in accordance with certain embodiments of the present disclosure.

FIG. 13 depicts a flow diagram of a method of selectively controlling radiation-emitting components of each therapy module, independently, in accordance with certain embodiments of the present disclosure.

FIG. 14 depicts a graphical interface including data and user-selectable control options, in accordance with certain embodiments of the present disclosure.

While implementations are described in this disclosure by way of example, those skilled in the art will recognize that the implementations are not limited to the examples or figures described. The figures and detailed description thereto are not intended to limit implementations to the form disclosed, but on the contrary, the intention is to cover all modifications, equivalents, and alternatives falling within the spirit and scope as defined by the appended claims. The headings used in this disclosure are for organizational purposes only and are not meant to limit the scope of the description or the claims. As used throughout this application, the word “may” is used in a permissive sense (in other words, the term “may” is intended to mean “having the potential to”) instead of in a mandatory sense (as in “must”). Similarly, the terms “include”, “including”, and “includes” mean “including, but not limited to”.

### DETAILED DESCRIPTION OF ILLUSTRATIVE EMBODIMENTS

Embodiments of systems, methods, and devices are described below that may include a system including one or more therapy modules and a control device, which may be integrated in one or more of the modules or which may communicate with one or more of the modules through a communications link. Each module may include an array of radiation-emitting components, such as light-emitting diodes (LEDs), organic LEDs (OLEDs), quantum dot LEDs (QLEDs), polymer LEDs (PLEDs), electroluminescence (EL) thin film coatings, radiation-emitting nanomaterials, other radiation-emitting components, magnetic field emitting components, electric field emitting components, other human-safe electromagnetic radiation components, or any combination thereof. In general, each module may be configured to emit electromagnetic radiation toward a patient.

The systems, methods, and devices may include a control device configured to send signals to each of the therapy modules to control the radiation-emitting components to selectively emit electromagnetic radiation. As used herein, the term “electromagnetic radiation” refers to light (ultraviolet, infrared, and visible), magnetic fields, electric fields, other electromagnetic radiation, or any combination thereof. As used herein, the term “electromagnetic radiation” also includes or refers to photons, which is a particle representing a quantum of light or other electromagnetic radiation. More generally, the radiation-emitting components may include any component configured to emit human-safe electromagnetic radiation at a selected wavelength and modulation (i.e., amplitude, duty cycle, duration, and timing) to provide a desired therapeutic effect. Such therapeutic effects may impact a selected treatment area and may include pain remediation, wound sterilization, inflammation reduction, stimulation of collagen production, enhanced blood flow, other therapeutic effects, or any combination thereof.

In some implementations, each therapy module may include a device interface configured to communicate with one or more of the control device or a computing device. Each therapy module may include an array of radiation-emitting components and a waveform generator configured to generate one or more waveforms to drive one or more of the radiation-emitting components of the array to emit electromagnetic radiation toward the patient to provide a selected physiological effect. The waveform generator may be configured to generate a variety of waveform signal shapes to drive the radiation-emitting components. The waveform signals may include one or more of a square wave signal, a triangular wave signal, a ramp wave signal, a sine wave signal, a sawtooth wave signal, or another wave shape. The waveform generator may be configured to generate periodic waveforms and aperiodic waveforms. In some implementations, the waveform generator may be configured to selectively control the wavelength and the modulation (the timing, the periodicity, the duty cycle, the pulse widths, the duration, the amplitude, and other parameters) of the signals that drive the radiation-emitting components.

In some implementations, each therapy module may modulate the output of each of its radiation-emitting components independently in response to signals from the control device or from an integrated controller, such as a microcontroller unit (MCU) or another processor. In some implementations, each therapy module may receive a signal from the control device, select one or more of pattern data or waveform data (or instructions) stored in a memory of the therapy module based on the received signal, and modulate the output of one or more of its radiation-emitting components based on one or more of the pattern data or the waveform data. The waveforms may operate as drive signals for the radiation-emitting components such that a change in frequency or amplitude of the drive signals may impact the wavelength and modulation of the electromagnetic radiation output.

In some implementations, a controller (MCU or processor) of the therapy module may control the entire array of radiation-emitting components using a selected waveform and according to one or more of a selected pattern or a dynamically determined pattern. In some implementations, the microcontroller may be configured to utilize artificial intelligence (AI) and machine learning (ML) to dynamically determine one or more patterns based on the sensor data. In other implementations, the microcontroller may communicate data through a network to an analytics system, which may be configured to dynamically determine one or more patterns by using one or more of an artificial intelligence system or machine learning to process the sensor data. The analytics system may communicate the determined one or more patterns to the microcontroller, which may apply them for delivering electromagnetic radiation to the patient.

In other implementations, the controller of the therapy module may control one or more subsets of the array of radiation-emitting components using one or more selected waveforms and according to one or more selected patterns. In still other implementations, the controller may control each emitting component independently such that each emitting component may be controlled by a selected waveform and according to a selected pattern, which may be different from some or all the rest of the other radiation-emitting components of the array. In some implementations, the controller may cause the waveform generator to provide different waveform shapes at the same or different wavelengths to one or more radiation-emitting components of the array. In some implementations, the controller may cause the waveform generator to modulate the waveform shape, the timing, the amplitude, the frequency, other parameters, or any combination thereof to selected radiation-emitting components individually, in subsets, or altogether, thereby varying the electromagnetic radiation output.

In some implementations, the therapy module may include or be coupled to one or more sensors that may be configured to generate signals indicative of one or more parameters of a user, such as blood flow, temperature, blood sugar, chemical contents of sweat, birefringence in coherent structures of living tissue, other parameters, or any combination thereof. In some implementations, the sensors may be configured to determine parameters of the user's skin, soft tissue, muscle, bone, fascia, and so on. In some implementations, the sensors may be configured to determine the presence of microbes; enzymes; dirt, particles, or debris; parasites; chemicals; viruses; other contaminants; or any combination thereof. In some implementations, one or more of the sensors may be configured to measure birefringence, polarization (a polarization sensor), or other sensors configured to determine organization of organic tissue and optionally inorganic materials. “Structured water” (sometimes referred to as “stable water”, “polywater”, “exclusion zone water” or “EZ water”, “4th phase of water”, or “gel/water matrix”), which may behave like a liquid crystal similar to fascia. The one or more sensors may be configured to determine the structure of the water based in part on the sensed organization of the biological materials, which may be reorganized into stable molecules. In some implementations, unstable or disorganized structures may be indicative of pain or injury, and changes in the organization of the biological materials may be an indication of the efficacy of a particular treatment pattern. The therapy module may include a memory configured to store data indicative of the determined parameters, and the therapy device may communicate the stored data to a control device.

The sensors may include resistive sensors, capacitive sensors, optical sensors, radiant sensors, biosensors, chemical sensors, polarization sensors, birefringence sensors, other sensors, or any combination thereof. The therapy device may be configured to selectively adjust signals provided to one or more of individual radiation-emitting components, subsets of radiation-emitting components, or arrays of radiation-emitting components based in part on the signals from the one or more sensors. In an example, the therapy device may be configured to facilitate and enhance blood flow, the one or more parameters monitored by the sensors may include blood flow, and a controller of the therapy device may be configured to selectively control one or more of the radiation-emitting components or alter a waveform pattern based on the blood flow sensor data to enhance the therapeutic effect.

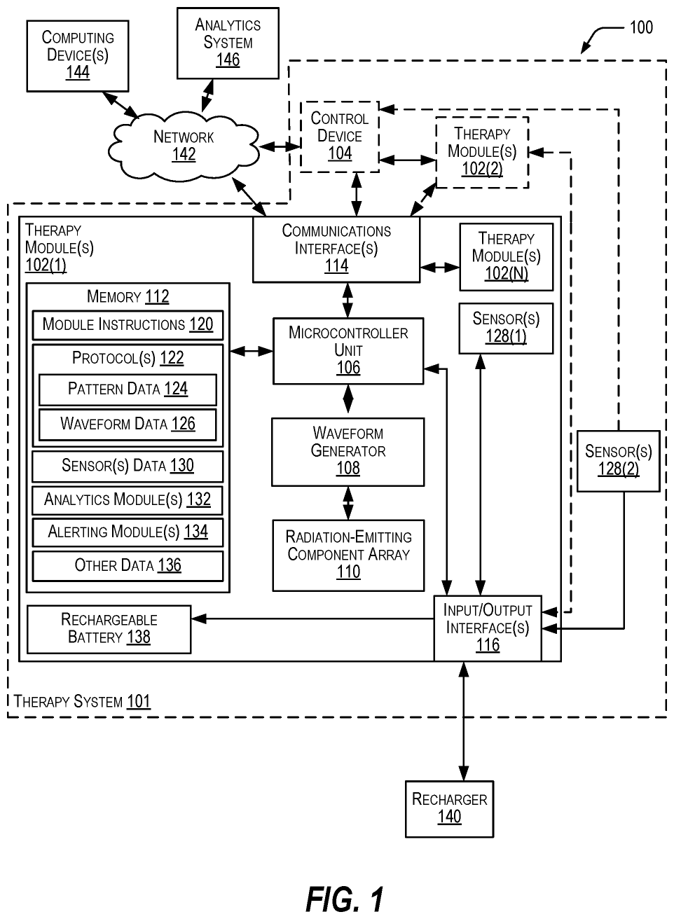

FIG. 1 depicts a block diagram of a system 100 including a therapy system 101 comprised of a plurality of therapy modules 102 and a control device 104, in accordance with certain embodiments of the present disclosure. The control device 104 may be configured to communicate with one or more of the therapy modules 102 by a wired connection or a wireless short-range radio frequency communications link or through a communications network 142, such as a local area network, a wide area network, a Wireless Fidelity (Wi-Fi) network, the Internet, other networks, or any combination thereof. The control device 104 may be implemented as a hand-held controller or as an application executing on a computing device, such as a smartwatch, a smartphone, a tablet computer, a laptop computer, or another computing device. In some implementations, the control device 104 may be integrated in a control system within an ambulance or other patient transport vehicle and may be coupled to one or more therapy modules 102 via one or more communications interfaces, which may include wired connections, wireless (radio frequency or optical) communications, or any combination thereof.

The therapy system 101 may include one or more therapy modules 102, which may be mechanically coupled or distributed and which may be communicatively coupled to provide a therapy system 101 having selected configurations and coverage areas. The therapy modules 102 may be formed from a flexible material that may conform to the surface shape of the patient. In addition to therapy modules 102 that are coupled together mechanically and communicatively, the therapy system 101 may also include one or more therapy modules 102 that are mechanically separated from the assembled therapy modules 102 but that may communicate with the other therapy modules 102 and optionally with the control device 104 through one or more wired or wireless communications links. Each therapy module 102 may include a microcontroller unit (MCU) or a processor 106 configured to execute instructions and optionally process data from one or more sensors 128. In some implementations, the MCU 106 may be implemented as a general purpose processor configured to process data and to execute processor-readable instructions.

The therapy module 102 may include a waveform generator 108, which may be configured to generate a plurality of driver signals to drive radiation-emitting components of an array 110. The waveform generator 108 may produce the driver signals in response to control signals from the MCU 106. The driver signals from the waveform generator 108 may be configured to control one or more parameters of the electromagnetic radiation emitted by the radiation-emitting components of the array 110, such as the intensity, waveshape, wavelength, duration, and timing of the emitted electromagnetic radiation. In some implementations, the waveform generator 108 may be configured to produce a driver signal for each emitting component of the array 110 or for subsets of the radiation-emitting components of the array 110, depending on a treatment protocol or depending on the implementation. The emitting component array 110 may be configured to emit human-safe electromagnetic radiation toward the user's skin to provide a selected therapeutic treatment. As used herein, “human-safe electromagnetic radiation” refers to light (ultraviolet, infrared, or visible) including photons, magnetic fields, electric fields, other radiation, or any combination thereof that is at a power level, a frequency level, and a duration that is insufficient to cause damage to the patient.

The therapy module 102 may include a memory 112, which may include one or more non-volatile memory devices. The memory 112 may be configured to store instructions 120 that may be executed by the MCU 106 to control operation of the therapy module 102 including receiving instructions from the control device 104 through one or more communications interfaces 114. The one or more communications interfaces 114 may include one or more connectors to support a wired connection or one or more transceivers configured to enable wireless radio frequency communications. The one or more communications interfaces 114 may enable a communications link between the therapy module 102 and the control device 104, between the therapy module 102(1) and one or more other therapy modules 102(2), between the therapy module 102 and one or more computing devices 144 through a communications network 142, between the therapy module 102 and an analytics system 146, or any combination thereof. The one or more computing devices 144 may include a desktop computer, a tablet computer, a laptop computer, a smartphone, another computing device, or any combination thereof.

Each therapy module 102 may include one or more rechargeable batteries 138, which may supply power to the various components. The therapy module 102 may include one or more input/output (I/O) interfaces 116. The one or more I/O interfaces 116 may include a port or connector configured to receive a cord or connector of a recharger 140 to recharge the batteries 138 and optionally to power the therapy system 101. In some implementations, the I/O interfaces 116 may include an inductive charge circuit configured to receive an electrical charge from an inductive recharger 140. In some implementations, the therapy module 102(1) may be electrically and optionally mechanically coupled to one or more adjacent therapy modules 102(2) by one or more wires extending from the I/O interface 116 of the therapy module 102(1) to the one or more adjacent therapy modules 102(2). In some implementations, the therapy modules 102 may share power between one another.

The therapy module 102 may communicate with one or more other therapy modules 102 and one or more sensors 128 through one or more input/output (I/O) interfaces 116. The one or more I/O interfaces 116 may include wired connections configured to communicatively couple the therapy module 102(1) to one or more other therapy modules 102 to send control signals, to share sensor data, to share power, or any combination thereof.

The memory 112 may include one or more protocols 122 that may be executed by the microcontroller unit 106 to control the waveform generator 108 to provide signals to the emitting component array 110 according to a selected one of the protocols 122. The protocols 122 may include pattern data 124, which may include a plurality of patterns relating to time-varying parameters, such as timing and duration as well as other variations in the waveform, such as the shape, the amplitude, the wavelength, the duty cycle, other parameters, or any combination thereof. The protocols 122 may also include waveform data 126, which may include a plurality of waveform shapes, such as a sine wave, a square wave, a ramp wave, a sawtooth wave, an irregular wave, other wave forms, or any combination thereof, which may be periodic or aperiodic. The protocols 122 may be stored in the memory 112. In some implementations, the memory 112 may include a plurality of protocols 122, which may include default protocols and customized protocols. Additionally, the user may modify one or more of the default protocols 122 and rename them to provide customized protocols 122.

In some implementations, the memory 112 may include instructions that may cause the microcontroller unit 106 to process sensor data from the sensors 128 relative to the applied protocol 122 and to dynamically adjust one or more parameters of the protocol 122 to enhance a selected physiological outcome. For example, if a selected physiological effect involves increased or improved blood flow of the patient, the microcontroller unit 106 may determine that the blood flow associated with one of the therapy modules 102 has reached a selected blood flow level and may reduce the emitted radiation from that therapy module 102 while adjusting parameters of other therapy modules 102 to emit electromagnetic radiation to increase blood flow at another location.

In other implementations, the memory 112 may include instructions that may cause the microcontroller unit 106 to process the sensor data from the sensors 128 relative to the applied protocol 122 (and optionally relative to historical data for the patient (or for all patients)) and may dynamically or automatically generate adjustments or even generate a new protocol 122 based on the data. The new protocol 122 may include an adjusted version of an existing protocol 122 or may be generated new from scratch.

In some implementations, the microcontroller unit 106 may communicate data including sensor data and protocol data to an analytics system 146 through the network 142. The analytics system 146 may apply machine learning, artificial intelligence, neural networks, filters, or any combination thereof to dynamically generate protocol adjustments or to dynamically generate a new protocol 122. The analytics system 146 may communicate the protocol adjustments to the therapy modules 102, which may implement the protocol adjustments. In some implementations, the analytics system 146 may push new protocols 122 to the therapy modules 102, which may store them in memory 112.

In some implementations, the memory 112 may store sensor data 130, which may include data captured from the one or more sensors 128. In some implementations, the therapy modules 102 may store sensor data 130 in their respective memories 112 and may selectively communicate the sensor data 130 to a control device 104 or to one or more of a computing device 144 or the analytics system 146 through the network 142. The memory 112 may also store other data 136, which may include usage log data, selected protocol data, patient data, and other data.

The memory 112 may include one or more analytics modules 132 that may cause the MCU 106 to analyze sensor data from the one or more sensors 128 relative to the selected protocol 122 to determine the efficacy of the selected protocol 122. In some implementations, the analytics modules 132 may cause the MCU 106 to dynamically adjust one or more parameters or characteristics of the selected protocol 122 based on the sensor data. In some implementations, the analytics modules 132 may cause the MCU 106 to dynamically generate new or derivative treatment protocols 122 based in part on the selected protocol 122 and the sensor data and may store the new or derivative treatment protocols 122 in the memory 112. In some implementations, the analytics modules 132 may cause the MCU 106 to determine a medical issue relating to the patient, based on the sensor data.

The memory 112 may include one or more altering modules 134 that may cause the MCU 106 to automatically generate an alert to one or more of the control device 104, a computing device 144, or the analytics server 146 in response to determination of a medical issue by the analytics modules 132. The alert may include text, sensor data, and information related to the medical issue so that the person administering the treatment or appropriate medical personnel are made aware of the medical issue.

The therapy module 102 may be configured to implement a selected one or more protocols 122 by controlling the waveform generator 108 to provide one or more signals to the emitting component array 110. The signals may cause the emitting component array 110 to emit human-safe electromagnetic radiation having a selected intensity, a selected amplitude, a selected wavelength, a selected timing, and a selected duration according to the selected protocol 122.

In some implementations, the therapy module 102 may include one or more integrated sensors 128(1), may be coupled to one or more external sensors 128(2), or any combination thereof. The sensors 128 may include resistive sensors, capacitive sensors, optical sensors, radiant sensors, biosensors, chemical sensors, other sensors, or any combination thereof. The sensors 128 may be configured to generate signals indicative of one or more of contaminants on a patient or physiological parameters associated with the patient. The contaminants may include microbes; enzymes; dirt, particles, or debris; parasites; chemicals; viruses; other contaminants; or any combination thereof. The medial parameters may include data related to the user's skin, soft tissue, muscle, bone, fascia, blood flow, temperature, blood sugar, chemical contents of sweat, other parameters, or any combination thereof. The sensors 128 may be configured to generate signals indicative of capillary activity, endothelium tissue activity, lymph activity, parameters of the fascia, parameters of bone marrow, parameters associated with overall well-being, other parameters, or any combination thereof.

In some implementations, the one or more sensors 128 may include an optical sensor configured to generate electrical signals indicative of coherent structures in the patient's tissue (e.g., skin, fascia, muscle, etc.). In particular, the sensor 128 may be configured to generate electrical signals indicative of birefringence of the tissue, which may be indicative of structured water molecules aligned to the coherent structures of the tissue. Sensor signals indicative of a high level of birefringence may be indicative of effectiveness and extent of the patient's response to the selected treatment. In contrast, misalignment of the water molecules may be indicative of incoherent or irregular structures in the tissue, which may indicate pain or injury. The microcontroller unit 106 may control the therapy modules 102 according to a selected protocol 122 and may optionally adjust one or more parameters (or even change the protocol 122) based on the birefringence data and other sensor data from the sensors 128.

In some implementations, the therapy module 102 may be configured to receive an input from the control device 104, retrieve a selected protocol from the protocols 122 in the memory 112 based on the received input, and generate one or more waveforms using the waveform generator 108 to selectively activate one or more radiation-emitting components of the component array 110 according to the selected protocol 122. The MCU 106 may be configured to monitor one or more physiological parameters associated with a user based on sensor signals from one or more of the sensors 128 and may selectively control the waveform generator 108 to adjust signals (or may change the protocol 122 for the waveform generator 108) or to produce associated signals that may be provided to one or more of the radiation-emitting components of the array 110 based on the sensor signals to achieve a selected physiological effect.

In an example, the selected protocol 122 may be configured to control the radiation-emitting components of the array 110 to emit electromagnetic radiation that varies over time with respect to one or more of a selected wavelength, a selected peak amplitude, a selected duty cycle, a selected timing, or a selected duration to enhance blood flow in a treatment area or in nearby areas on a patient's body. In an example, one or more of the sensors 128 may monitor blood flow and changes in the blood flow over time (based on sensor data 130 from the one or more sensors 128) as the therapy module 102 emits electromagnetic radiation toward the treatment area. In some implementations, one or more of the sensors 128 may be configured to determine birefringence of water or of the patient's tissue and to determine changes in the birefringence in the treatment area on the patient's body. The MCU 106 may automatically adjust one or more of the intensity, the wavelength, the timing, the duration, another characteristic, or any combination thereof based on the sensor data to provide a selected therapeutic effect, such as enhanced blood flow in the treatment area and related areas, pain remediation, wound healing, or other therapeutic effects. The sensors 128 may continue to provide sensor signals, and the MCU 106 may selectively adjust one or more characteristics of the emitted human-safe electromagnetic radiation in response to the sensor signals.

In some implementations, variability of selected characteristics of the emitted electromagnetic radiation may be part of the selected protocol 122. The characteristics of the selected protocol 122 may include an on/off pattern, a time-based variation in one or more of the wavelength or the amplitude of applied electromagnetic radiation, a time-based variation in the shape of the waveform that drives the radiation-emitting components (e.g., sine wave, sawtooth wave, square wave, triangular wave, ramp wave, irregular wave, or another waveform). The selected protocol (its various characteristics) may be customized for the specified treatment, for the specified treatment area, for the size of the treatment area of the patient, based on parameters of the patient (height, weight, body fat, skin pigmentation, other parameters, etc.), based on other factors, or any combination thereof.

An operator may arrange the therapy system 101 in a selected configuration by adding therapy modules 102 to or removing therapy modules 102 from the patient. The therapy system 101 may include any number of therapy modules 102 coupled together in a selected configuration or distributed individually across a treatment area. In the illustrated example, a therapy system 101 may be comprised of a plurality of therapy modules 102. In some applications, two or more of the therapy modules 102 may be coupled to together along adjacent edges to form a larger therapy module 102 and other therapy modules 102 may be distributed across the treatment area In some implementations, the control device 104 may establish a communications link to one or more of the therapy modules 102, such as the therapy module 102(1). The control device 104 may communication with other therapy modules 102(2) through 102(N) through the first therapy module 102(1) or directly via a separate communications link. In some implementations, the first therapy module 102(1) that establishes communication with the control device 104 may be a “master” module, and the other therapy modules 102 that connect to the control device 104 through the first therapy module 102(1) may be “slave” modules for the purposes of timing and control. In other implementations, each therapy module 102 may operate independently and the control device 104 may address control signals directly to a selected therapy module 102 either via a direct connection or an indirect connection through an intervening module 102. In a master-slave configuration that uses a daisy-chain communication path from the control device 104 through other therapy modules 102, the therapy module 102(N) may be responsive to control signals addressed to it and may ignore other control signals that are addressed to other therapy modules 102.

In an example, the other data 136 may include an identifier configured to uniquely identify the therapy module 102(1) relative to other therapy modules 102. The module instructions 120 may cause the MCU 106 of one or more of the therapy modules 102 to determine the relative configuration of the various therapy modules 102 that are connected along the edges to form an assembled therapy module 102. When activated, the MCU 106 may determine the I/O interface 116 to which a therapy module 102 is connected, determine the identifier of the therapy module 102, and determine a map or configuration of the assembled therapy module 102 based on the identifiers and the associated I/O interface 116. The individual therapy modules 102 within the assembled group may communicate with one another to determine interconnection data that may be used to determine a configuration or arrangement of the therapy modules 102 within the assembled group. The interconnection data may be communicated by the first therapy module 102(1) to the control device 104, which may process the received interconnection data to determine the physical position of each of the therapy modules 102 that form the assembled group. The control device 104 may utilize this information to send control signals and timing data to the therapy modules 102 to implement one or more selected protocols 122 for the assembled group.

In some implementations, a first portion of the therapy system 101 (comprised of one or more therapy modules 102) may be configured to activate one or more radiation-emitting components of the respective one or more arrays 110 based on a first protocol 122 and a second portion of the therapy system 101 (comprised of one or more other therapy modules 102) may be configured to activate one or more radiation-emitting components of the respective one or more arrays 110 based on a second protocol 122. In other implementations, the selected protocol 122 may specify the waveform shapes, wavelengths, amplitudes, duty cycles, timings, and durations for each radiation-emitting component of the arrays 110 of the therapy modules 102, which may coordinate their operation based on the selected protocol 122 and based, at least in part, on their relative positions within the arrangement of therapy modules 102 that comprise the therapy system 101.

In some implementations, the therapy modules 102 may coordinate with one another to self-organize based on communications between the therapy modules 102 to form a therapy system 101 within which the therapy modules 102 may cooperate to work as a “team”. Each therapy module 102 may be controlled by the control device 104, independently, or as part of (in combination with) the array of therapy modules 102 that form the therapy system 101.

In an example, an operator may have a plurality of therapy modules 102 and may choose to couple six of the therapy modules 102 (mechanically and communicatively) to form a first assembled group and to use one or more therapy modules 102 separately to provide a therapy system 101 to target at least two or more areas of the body simultaneously and with the same or different protocols 122. In this implementation, the assembled group of therapy modules 102 and the one or more other (individual or groups of) therapy modules 102 may communicate with one another and may coordinate with one another to provide a selected therapeutic effect.

In another implementation, a first assembled group of therapy modules 102 may be used in conjunction with other therapy modules 102 (alone or in a second assembled group) to apply electromagnetic radiation to two or more areas of the body simultaneously and with the same or different protocols 122. In this example, the first assembled group of therapy modules 102 and the other therapy modules 102 may operate independently or in a coordinated manner using one or more control devices 104.

The control device 104 may be configured to control one or more radiation-emitting components, subsets of radiation-emitting components, or the array 110 of radiation-emitting components of each therapy module 102. In some implementations, the MCU 106 of a therapy device 102 or the control device 104 may selectively adjust one or more parameters of a selected protocol 122 based on sensor data from one or more sensors 128 to achieve a selected physiological or therapeutic effect, such as increased blood flow in a desired area, birefringence data, other data, or any combination thereof.

In some implementations, the control device 104 may monitor data corresponding to the applied electromagnetic radiation, such as total energy dosage, density, duration, and other data. The control device 104 or the MCU 106 of a therapy module 102 may turn off one or more radiating components of the array 110 (individually, in subsets, or in total) while continuing with the protocol at other therapy modules 102. In a non-limiting example, an energy dosage of two Joules/centimeter-squared (2 J/cm²) may be deemed a “low dose” while an energy dosage of 16 J/cm² may be deemed a “high dose.” The range of low and high may vary based on characteristics of the patient, based on the treatment area, based on the type of treatment, or based on other factors. In some implementations, the dosage levels may be monitored for safety purposes, and the therapy modules 102 may turn off one or more of the radiation-emitting components of the array 110 when the dosage exceeds a threshold level, which may be set by an operator or which may be preconfigured during programming.

In some implementations, the control device 104 may be configured to aggregate data corresponding to measured parameters one or more patients or users. Subsequently, the control device 104 may analyze the data for a patient or user or across a plurality of patients or users. In other implementations, the control device 104 may communicate data to an analytics system 146 for further processing and analysis. In an example, the patient or user data may be processed to remove personally identifying information (PII) prior to providing the data to the analytics system 146. The analytics system 146 may process received data to determine adjustments to existing protocols 122 or to determine new protocols 122 based on data indicative of the response of multiple patients to signals applied to the arrays 110. Other implementations are also possible.

In some implementations, one or more therapy modules 102 may be applied to a user, such as on the user's forearm, shoulder, wrist, or another location on the user's body. Additionally, one or more therapy modules 102 may be configured to interact with an existing wearable device (i.e., control device 104), such as a smartwatch or other device, applying one or more protocols to the user while providing sensor data to the control device 104, which may incorporate the sensor data into its aggregated data and which present the sensor data together with other data it has collected. For example, the therapy module 102 may provide circulation data (data indicative of blood flow) to the control device 104, which may provide the circulation data together with heart rate, sleep information, activity monitoring data, and other data, either on its display or within an application executing on another device, such as a smartphone.

In some implementations, the therapy module 102 may be configured to generate sensor data from the one or more sensors 128 that may be indicative of blood flow, which may be used to detect circulatory issues, such as impingements, blood clots, or other issues. In some implementations, the sensor data 128 may include birefringence data indicative of alignment of water molecules and tissue structures. The therapy modules 102 may be useful for monitoring parameters of military personnel, athletes, and other individuals in high-stress environments or in arduous situations. In some implementations, the therapy modules 102 may be deployed along the neck and spine and the sensors 128 may produce data indicative of neurological impulses below the brainstem and optionally within the brain.

In some implementations, the therapy modules 102 may be utilized for transcranial applications in the treatment of various neurological impairments, including dementia, traumatic brain injury, or diseases (such as Parkinson's disease, Alzheimer's disease, and other diseases). The sensors 128 may be configured to monitor capillary activity, endothelium tissue activity, lymph systems, fascia, bone marrow, and other parameters of the patient.

The therapy modules 102 may be used in conjunction with other fitness or well-being applications or systems, such as smartwatches, fitness bands, or other devices and their associated applications, which may be executed on a smartphone or computing device. The therapy modules 102 may provide sensor data that may supplement information determined by the fitness or well-being systems and that may enable enhanced analytics that may be used to improve a fitness program.

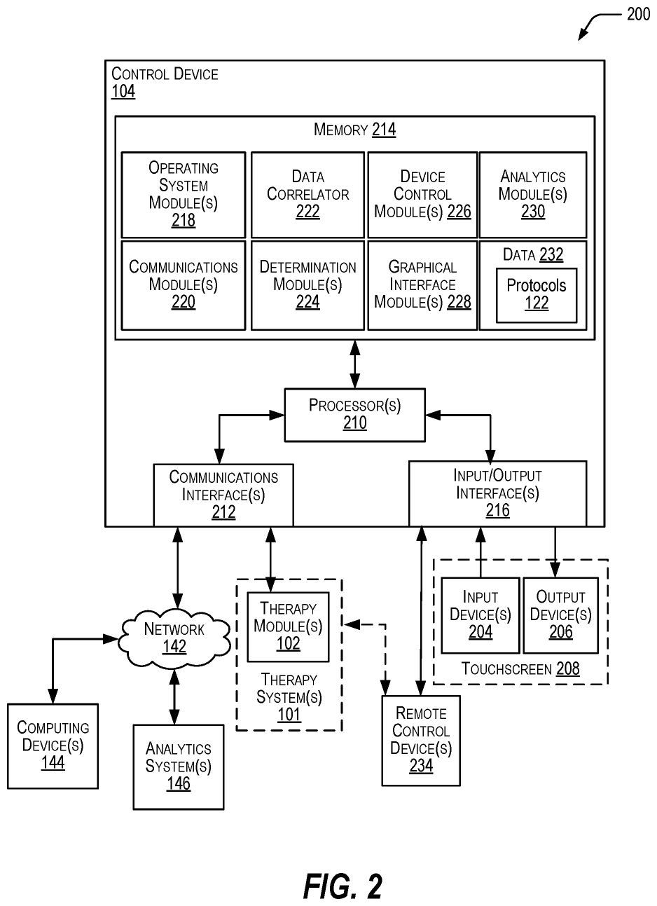

FIG. 2 depicts a block diagram of a system 200 including an implementation of the control device 104 of FIG. 1, in accordance with certain embodiments of the present disclosure. The control device 104 may be a computing device, such as a smartphone, a tablet computer, a laptop computer, or another computing device. In some implementations, the control device 104 may be integrated into a larger device, such as an instrument console of an ambulance. In other implementations, the control device 104 may be implemented as a hand-held electronic device, such as a television remote control or other control device including one or more user-selectable control options.

The control device 104 may be configured to communicate with one or more therapy modules 102 via wired connections or via one or more short-range wireless radio frequency communications links. The control device 104 may be configured to communicate with an analytics system 146, with one or more other computing devices 144, or any combination thereof through a communications network 142, such as the Internet, a short-range network, another type of network, or any combination thereof.

The control device 104 may include or may be coupled to one or more input devices 204 and one or more output devices 206. The input devices 204 may include a keypad, a touch-sensitive interface, a mouse, a stylus, a microphone, a camera, a scanner, other input devices, or any combination thereof. The output devices 206 may include a display, a speaker, a printer, another output device, or any combination thereof. In some implementations, a touch-sensitive interface of the input devices 204 and a display of the output devices 206 may be combined in the form of a touchscreen 208. In some implementations, the control device 104 may be responsive to control signals from a remote control device 234 through a wired or wireless communications link. The remote control device 234 may include one or more of buttons, switches, a display, a touch-sensitive interface, or other user-selectable controls. In an example, the remote control device 234 may be configured to control one or more of the control device 104 or the therapy system 101.

The control device 104 may include a processor 210 configured to execute instructions, which may be stored in a memory 214 that is coupled to the processor 210. The memory 214 may include one or more non-volatile memory devices configured to store instructions and data.

The control device 104 may include one or more communication interfaces 212 coupled to the processor 210. The communication interfaces 212 may include wired connections and radio frequency transceivers configured to send and receive data via wireless radio frequency communications links. The communications interfaces 212 may include Ethernet connectors, Universal Serial Bus (USB) connectors, radio frequency transceivers configured to support short-range radio frequency communications (such as Bluetooth® protocols, Wi-Fi, IEEE 802.11x protocols, other short-range wireless protocols, or any combination thereof).

The control device 104 may include one or more input/output (I/O) interfaces 216, which may include connectors, such as USB connectors, a tip-ring-sleeve (TRS) connector, other connectors, or any combination thereof. In some implementations, the I/O interfaces 216 may include short-range radio frequency transceivers configured to communicatively couple the control device 104 to one of an input device 204 (such as a wireless keyboard or mouse) or an output device 206 (such as a wireless speaker).

The memory 214 may include one or more operating system modules 218 that may be executed by the processor 210 to control overall operation of the control device 104. The operating system modules 218 may include drivers for the communications interfaces 212 and for the I/O interfaces 216. The memory 214 may also include one or more communication modules 220 that may cause the processor 210 to send instructions to and receive data from one or more therapy devices 101 or to send data to and receive data from an analytics system 146 through the network 142 via the communication interfaces 212.

The memory 214 may include one or more data correlators 222 that may cause the processor 210 to receive sensor data from one or more of the therapy modules 102 of a therapy system 101. The data correlators 222 may cause the processor 210 to correlate received sensor data with treatment protocol data, timing data, patient data, and other data.

The memory 214 may include one or more determination modules 224 that may cause the processor 210 to receive signals from the one or more therapy modules 102 of the therapy system 101 that may be indicative of the relative positions of the various therapy modules 102 of the therapy system 101. The determination modules 224 may cause the processor 210 to determine identifier information associated with the therapy module 102 with the relative position of the therapy module 102 within the therapy system 101 to determine a mapping of the configuration of therapy modules 102.

The memory 214 may include one or more device control modules 226 that may cause the processor 210 to send signals to one or more of the therapy modules 102. The signals may be used to select and optionally configure the protocol 122 that may be implemented by the one or more therapy modules 102 or to selectively control one or more radiation-emitting components of one or more of the arrays 110. In some implementations, the device control modules 226 may cause the processor 210 to generate signals to selectively control individual radiation-emitting components of the arrays 110.

The memory 214 may include one or more graphical interface modules 228 that may cause the processor 210 to generate a graphical interface that may be provided to an output device 206 (such as a display). The graphical interface may include text, images, user-selectable control options (such as buttons, clickable links, pull-down menus, tabs, checkboxes, text fields, or other control elements), or any combination thereof.

The memory 214 may include one or more analytics modules 230 that may cause the processor 210 to process sensor data received from the one or more therapy modules 102, determine adjustments based on the sensor data and the selected protocol 122, and selectively adjust one or more of the radiation-emitting components or the protocol 122. In some implementations, the analytics modules 230 may cause the processor 210 to process sensor data from the one or more therapy modules 102 and, based on the sensor data, to generate new protocols 122, which may be pushed to the one or more therapy modules 102. In some implementations, the analytics modules 230 may selectively scrub personally identifying information from the sensor data and may send the scrubbed data to an analytics system 146 for further processing.

In some implementations, the processor 210 may store received data in the memory 214 as data 232. The data 232 may be correlated to the therapy module 102, the patient, the time, other data, and so on. The control device 104 may aggregate data over time for each patient and may store the aggregated data in the data 232. In some implementations, the data 232 may include a plurality of treatment protocols 122 that may be provided to the therapy system 101 to provide a selected treatment.

In some implementations, the analytics module 230 may be configured to process the aggregated data for a patient from the data 232. The analytics module 230 may utilize machine learning, neural networks, artificial intelligence, fuzzy logic, or any combination thereof to process correlated data for a patient and for a selected treatment protocol 122. Over time, the analytics module 230 may determine adjustments for the protocol 122 based on the patient's response to the emitted radiation. The analytics module 230 may communicate the adjustments directly to one or more therapy modules 102 or to the graphical interface modules 228 for presentation within a graphical interface provided to the output device 206 or to a computing device 144.

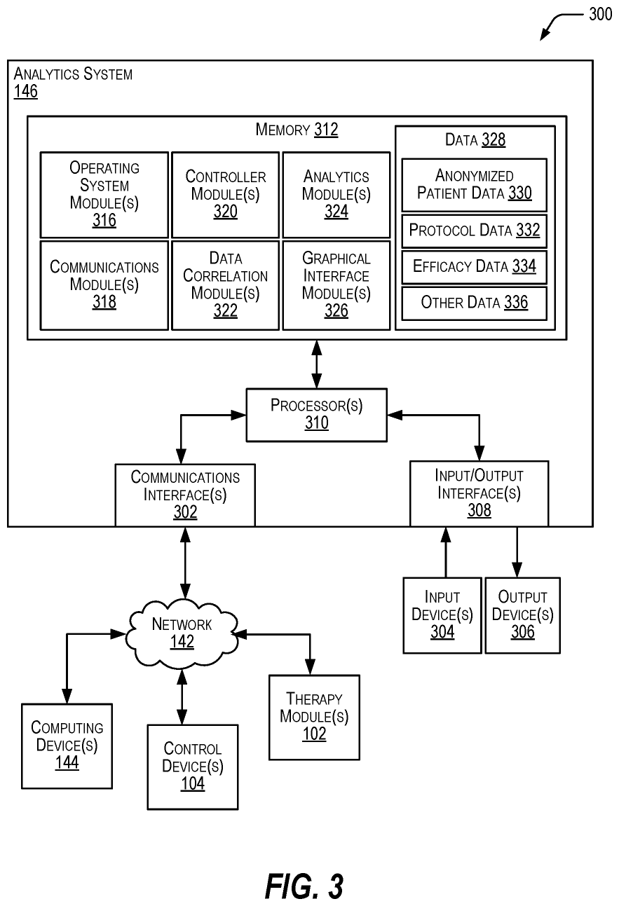

In some implementations, the analytics system 146 may be configured to generate new protocols 122 and to modify existing protocols 122 based on anonymous patient data. The analytics system 146 may be configured to process the data across multiple patients, multiple therapy systems 101, and over time to determine new or optimal protocols 122. The analytics system 146 may push new protocols 122 or updated protocols 122 to one or more control devices 104 and optionally to the therapy modules 102. An example of an analytics system 146 is described below with respect to FIG. 3.

FIG. 3 depicts a block diagram of a system 300 including an implementation of an analytics system 146 configured to communicate with one or more of computing devices 144 or one or more control devices 104 through the communications network 142, in accordance with certain embodiments of the present disclosure. The analytics system 146 may be implemented as a standalone computing device or may be distributed across multiple computing systems or server systems accessible via the network cloud.

In some implementations, the analytics system 146 may include one or more communications interfaces 302, which may be configured to communicate with one or more control devices 104 and one or more computing devices 144 through the communications network 142. The analytics system 146 may include one or more I/O interfaces 308, which may be coupled to one or more input devices 304 and to one or more output devices 306. The input devices 304 may include a keypad, a touch-sensitive interface, a mouse, a stylus, a microphone, a camera, a scanner, other input devices, or any combination thereof. The output devices 306 may include a display, a speaker, a printer, another output device, or any combination thereof.

The analytics system 146 may include a processor 310 coupled to the one or more communications interfaces 302 and to the one or more I/O interfaces 308. The processor 310 may also be coupled to a memory 312, which may store instructions and data. The memory 312 may include one or more operating system modules 316 that may be executed by the processor 310 to control operation of the various components, such as the I/O interfaces 308, other components, and so on.

The memory 312 may include one or more communications modules 318 that may cause the processor 310 to control operation of the communications interfaces 302 and to format data for communication to the control devices 104, to the computing devices 144, or any combination thereof.

The memory 312 may include one or more controller modules 320 that may cause the processor 310 to receive data from one or more control devices 104. The controller modules 320 may cause the processor 310 to send protocol data or other data to a selected control device 104. For example, over time, the analytics system 146 may determine one or more updated protocols 122 (based on analysis of data accumulated from multiple therapy systems 101 and the sensor data indicative of patients' responses to various protocols 122). In some implementations, the analytics system 146 may be configured to identify the relative effectiveness of various patterns, durations, timing, and other parameters of the protocols 122 based on the responsiveness of the patients' and may selectively adjust one or more of the protocols 122 based on the determined data.

The memory 312 may include one or more data correlation modules 322 that may cause the processor 310 to correlate the received data to an applied protocol, date data, efficacy data, a control device identifier, and other data received from the control device 104. The correlated received data may be stored as data 328 in the memory 312. The data 328 may include anonymized patient data 330, which may include the applied protocol, injury information, sensor data, other data, or any combination thereof without providing information that would be traceable to the patient. The data 328 may include protocol data 332, which may include baseline protocols and other protocols generated by the analytics system 146. The protocol data 332 may include default and dynamically generated protocols 122 that may have been provided to one or more of the therapy modules 102 as well as newly generated protocols 122 that have not yet been deployed. The data 328 may include efficacy data 334, which may include information about the impact of selected protocols and protocol adjustments on the patients. The data 328 may also include other data 336.

The memory 312 may include one or more analytics modules 324, which may be configured to process the anonymized patient data 330 to determine improvements to a protocol 122 based on the sensor data and the applied protocol 122. In some implementations, the one or more analytics modules 324 may cause the processor 310 to determine improvements to a protocol 122 for a particular patient from the anonymized patient data 330. The improvements may include time-based variations in amplitude, frequency, waveform type, or other characteristics of an electrical signal that drives the radiation-emitting components of the arrays 110. In other implementations, the one or more analytics modules 324 may cause the processor 310 to determine improvements to a protocol 122 based on anonymized patient data 330 from a variety of patients. The one or more analytics modules 324 may be configured, based on the anonymized patient data 330, to modify existing protocols 122 or to generate new protocols 122. In an example, over time, the analytics modules 324 may determine a point at which further exposure to the electromagnetic radiation either provides no benefit or marginal benefit or may cause damage, and the analytics modules 324 may set new thresholds or adjust existing protocols to prevent injury, conserve power, enhance a therapeutic benefit, and so on.

The memory 312 may include one or more graphical interface modules 326 that may cause the processor 310 to generate a graphical interface including images, text, user-selectable control options (i.e., buttons, tabs, clickable links, checkboxes, radio buttons, and other control elements), or any combination thereof. In some implementations, the user-selectable control options may include a first control option accessible by a user to access one or more existing protocols 122 and a second control option accessible by the user to edit one or more of the protocols 122. In some implementations, the graphical interface may include a third control option accessible by the user to create a custom protocol 122.

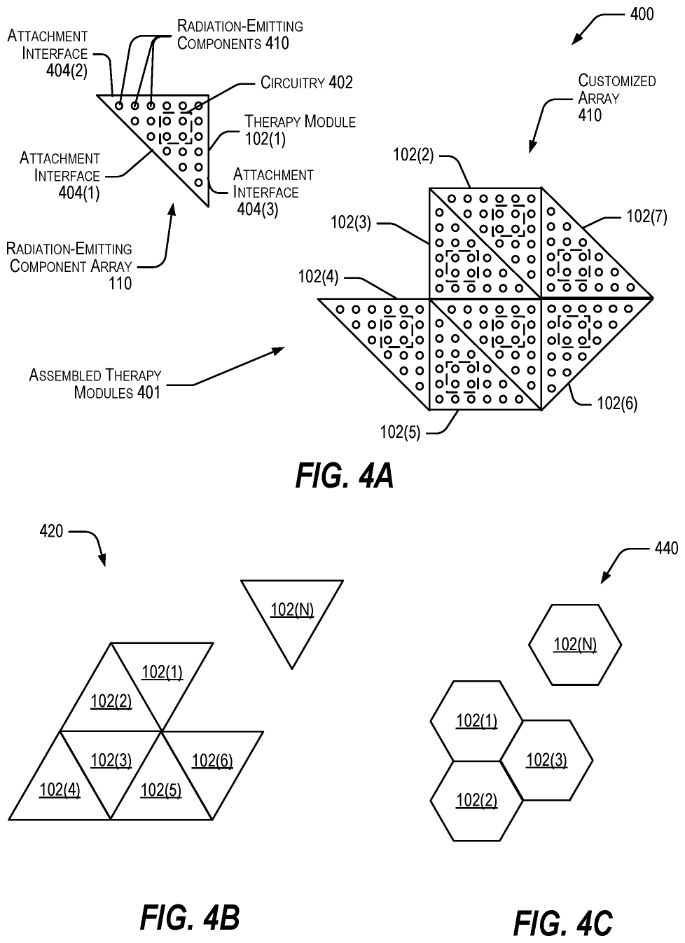

The therapy modules 102 may be formed from a flexible material that may conform to the surface shape of the patient. In some implementation, the therapy modules 102 may be implemented in a variety of form factors and shapes. An example of a modular implementation using right triangular-shaped therapy modules 102 is described below with respect to FIG. 4A.

FIG. 4A depicts a diagram of a system 400 including a modular therapy system 101 comprised of a plurality of right triangular-shaped therapy modules 102, in accordance with certain embodiments of the present disclosure. In this example, the therapy modules 102(2), 102(3), 102(4), 102(5), 102(6), and 102(7) are coupled together to form a therapy system 101. The therapy device 102(1) is not yet connected to the other modules 102. The system 400 may be operated in this configuration, for example, providing two therapy modules 102: therapy module 102(1) and the assembled therapy modules 401. Additional therapy modules 102 may also be included to provide a selected electromagnetic radiation therapy at multiple locations on the patient's body. In some implementations, the therapy modules 102 may be applied to a patient individually and may be spaced apart to allow movement by the patient and to apply therapeutic effects to selected areas of the patient's body.

Each therapy module 102 may include circuitry 402, including the MCU 106, the waveform generator 108, the radiation-emitting component array 110, the memory 112, the communications interface 114, the I/O interfaces 116, and the sensors 128. In addition to the array 110, the circuitry 402 may include or may be coupled to one or more sensors 128 (shown in FIG. 1), which may capture data indicative of one or more parameters. The sensors 128 may be distributed within the array 110, along the edges, or any combination thereof.

In this example, each therapy module 102 may include an attachment interface 404 on each side. Each attachment interface 404 may include an I/O interface 116 to facilitate interconnections between adjacent therapy modules 102. In some implementations, the attachment interfaces 404 may include physical connectors including physical communication ports to interconnect adjacent therapy modules 102. In other implementations, the attachment interfaces 404 may include communication interfaces configured to establish wireless (radio frequency) connections between therapy modules 102 to provide an ad hoc network of therapy modules 102. The communication interfaces may facilitate communication between the therapy modules 102 to provide a selected therapeutic effect. In some implementations, the therapy system 101 may be comprised of individual therapy modules 102, some of which may be coupled together and some of which may be spaced apart from others of the therapy modules 102. The therapy modules 102 may communicate with one another through wired connections (when available) and through wireless communications links when they are spaced apart.

The radiation-emitting component array 110 may include a plurality of radiation-emitting components 410 distributed across the surface area of the therapy module 102. In some implementations, the radiation-emitting components 410 may emit electromagnetic radiation through openings in a surface of the therapy module 102 or through a transparent surface of the therapy module 102, depending on the implementation.

Though the radiation-emitting components 410 are depicted as identical round shapes, the radiation-emitting components 410 may have other shapes, such as rectangular shapes, ellipse shapes, and so on. Within the radiation-emitting component array 110, the radiation-emitting components 410 may vary in size and shape such that a single radiation-emitting component array 110 may include smaller and larger radiation-emitting components 410 and rectangular, circular, elliptical, or other-shaped radiation-emitting components 410. In some implementations, the radiation-emitting components 410 may be formed from electrical components that may emit electromagnetic radiation including light (ultraviolet, infrared, and other wavelengths within the visible light spectrum), magnetic fields, electrical fields, photons, other electromagnetic radiation, or any combination thereof at selected frequencies, selected amplitudes, and selected duration. In some implementations, in lieu of or in addition to radiation-emitting elements, the array 110 may include one or more components configured to emit a time-varying magnetic field, a time-varying electrical field, or other time-varying electromagnetic radiation.

In some implementations, the right triangular-shape of the therapy modules 102 may enable a user to interconnect a plurality of therapy modules 102 to form a therapy system 101 having a selected shape to treat a selected area of the patient's body. The triangular-shaped therapy modules 102 may be interconnected to form square or rectangular-shaped systems 101, to form parallelogram-shaped systems 101, or to form other shapes. In this example, the right-triangle may be a right isosceles triangle shape, which may enable a wide range of interconnections and configurations. Other shapes are also possible.

FIG. 4B depicts a diagram of a system 420 of a modular therapy system 101 comprised of a plurality of equilateral-triangular shaped therapy modules 102, in accordance with certain embodiments of the present disclosure. In this example, some of the therapy modules 102 may be interconnected and one or more therapy modules 102 may be used independently to form one or more therapy systems 101 having selected shapes, configuration, and coverage areas.

FIG. 4C depicts a diagram of a system 440 including a modular therapy system 101 comprised of a plurality of hexagon-shaped therapy modules 102, in accordance with certain embodiments of the present disclosure. In this example, the therapy modules 102 may be interconnected to form at least a portion of a therapy system 101 having a selected shape. In some implementations, the therapy system 101 may include the interconnected modules 102 as well as one or more other (physically separate) therapy modules 102, which may be communicatively coupled to the interconnected therapy modules 102, such that the therapy system 101 may include both the interconnected therapy modules 102 and the physically separate therapy modules 102. Other shapes are also possible.

While the therapy systems 101 depicted in FIGS. 4A-4C are comprised of therapy modules 102 having the same shape (homogenous-shaped therapy modules 102), it is possible to form a therapy system 101 from therapy modules 102 having different shapes. In some implementations, such as the illustrated embodiments, the therapy modules 102 may have at least one straight edge to facilitate an interconnection. Alternatively, the therapy modules 102 may have edges that have a repeating shape, such as a sinusoidal waveform shape, a square waveform shape, a sawtooth shape, or another shape that repeats and that can be configured to couple to a corresponding shape of an edge of an adjacent therapy module 102. In other implementations, one or more of the therapy modules 102 may be implemented in a circular shape, and the therapy modules 102 may communicate with one another via wireless (radio frequency) signals.

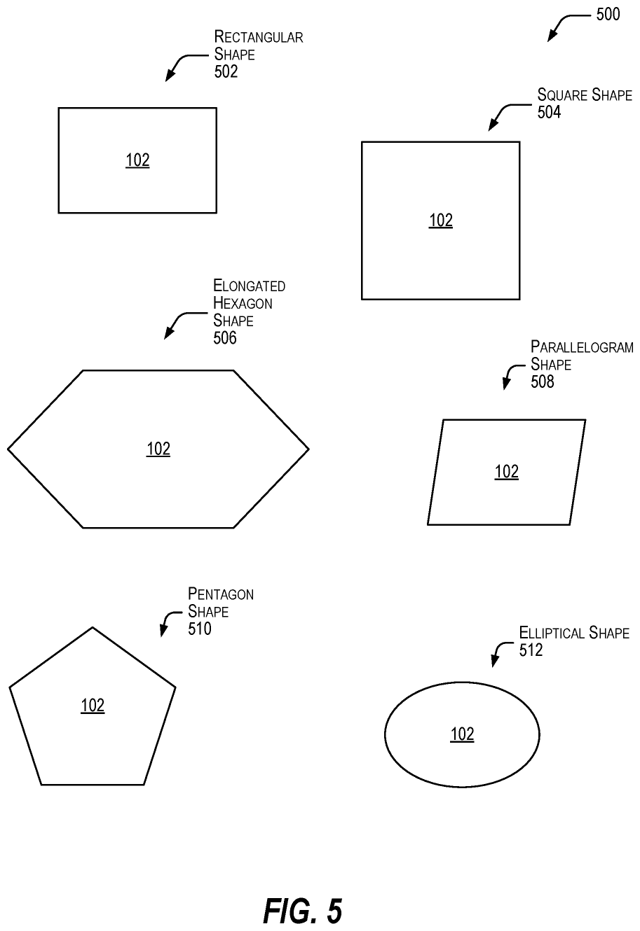

FIG. 5 depicts diagrams 500 of various shapes of modular therapy modules 102, in accordance with certain embodiments of the present disclosure. In this example, the therapy module 102 may have a rectangular shape 502, a square shape 504, an elongated hexagon shape 506, a parallelogram shape 508, a pentagon shape 510, or another shape. In the examples of FIGS. 4A-5, the various shapes have straight edges or corresponding-shaped edges to facilitate modular interconnections, allowing the user to group multiple therapy modules 102 to form the therapy system 101 having a desired configuration.

In some implementations, the therapy module 102 may be implemented in a circular or elliptical shape 512. In this configuration, the elliptical shape 512 may not be mechanically coupled to other therapy modules 102 along their edges, due to the shape. However, the elliptical shaped therapy modules 102 may be coupled mechanically and electrically by electrical conductors or may be communicatively coupled by wireless radio frequency signals.

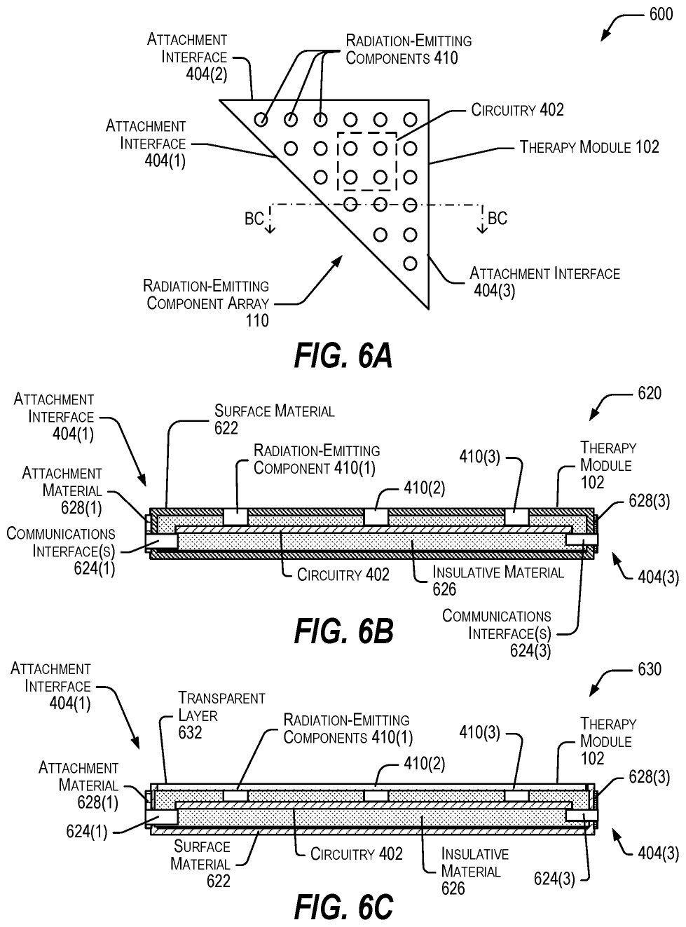

FIG. 6A depicts a diagram of a system 600 including a modular therapy module 102, in accordance with certain embodiments of the present disclosure. The modular therapy module 102 may include attachment interfaces 404 along each side. The attachment interfaces 404 may include hooks, hook and loop fabric components, magnets, or other mechanical attachment mechanisms configured to facilitate a mechanical interconnection between the therapy module 102 and another therapy module 102 along respective edges. In some implementations, the attachment interfaces 404 may include radio frequency transceivers, inductive interfaces, or other components configured to enable wireless recharging, wireless radio frequency communication, or any combination thereof, allowing the therapy modules 102 to be used as an array of modules 102 arranged close to one another but not necessarily physically connected to one another.

FIG. 6B depicts a cross-sectional view 620 of the therapy module 102 of FIG. 6A taken along line BC-BC in FIG. 6A, in accordance with certain embodiments of the present disclosure. In this view 620, the therapy module 102 may include a surface material 622 including openings through which the radiation-emitting components 410 may extend. The surface material 622 may form an enclosure sized to fit the circuitry 402 and insulative material 622 configured to surround the circuitry 402. The insulative material 626 may operate as padding to protect the circuit 402.

The surface material 622 may include the attachment interfaces 404(1) and 404(3) along its edges. The attachment interface 404(1) may include attachment material 628(1), and the attachment interface 404(3) may include attachment material 628(3). The attachment material 628 may include hook and loop fabric or another material configured to mechanically engage the corresponding material on an edge of an adjacent therapy module 102. In some implementations, the attachment interface 404 may also include one or more communications interfaces 624 configured to establish a communication path between adjacent therapy modules 402. The attachment interface 404(1) may include one or more communications interfaces 624(1), and the attachment interface 404(3) may include one or more communications interfaces 624(3). The communications interfaces 624 may include physical connectors, wireless communications interfaces, other interfaces, or any combination thereof.

In this example, the surface material 622 may be formed from antimicrobial material or from a material that may be wiped or otherwise cleaned after use. In some implementations, the therapy module 102 may include a resealable opening that may be accessed to remove the circuitry 402 and insulative material 626 so that the user may wash the surface material 622 without damaging the circuitry 402.

FIG. 6C depicts a cross-sectional view 630 of the therapy module 102 of FIG. 6A taken along line BC-BC in FIG. 6A, in accordance with certain embodiments of the present disclosure. In the view 630, the therapy module 102 may include all the elements of the therapy module 102 of FIG. 6B, except that the enclosure is formed by a combination of the surface material 622 and a transparent layer 632, which may be or which may include an adhesive (with or without medication). The transparent layer 632 may be formed from a material or composition that allows emitted radiation from the radiation-emitting components 410 to pass through.

In some implementations, the transparent layer 632 may be formed from a silicone gel composition that may operate as a reusable adhesive. In other implementations, the transparent layer 632 may be formed from or may include a topical medication configured to treat the surface of the skin or a transdermal medication configured to transport medication to and through the skin. Depending on the implementation, the transparent layer 632 may include painkillers, ointment, moisturizers, antibiotics, other medications, or any combination thereof.

In some implementations, the therapy module 102 may be applied to the patient using a transparent coating (such as a medicinal coating or a transparent bandage). In some implementations, the transparent coating may provide a double-sided adhesive functionality to secure the therapy module 102 to the patient. The medicinal coating or transparent bandage may facilitate selected therapeutic effects. Such therapeutic effects may impact a selected treatment area and may include pain remediation, wound sterilization, inflammation reduction, stimulation of collagen production, enhanced blood flow, and so on.

In some implementations, by deploying the therapy modules 102 such that they are spaced apart from one another and attached to the skin of the patient by the transparent layer 632. The spacing between therapy modules 102 may allow the patient to maintain a full range of motion while wearing the therapy modules 102 and receiving treatment.

In this example, the surface material 622 and the transparent material 632 may be readily cleaned by wiping the material with a cleaner after use. Additionally, the transparent layer 632 may protect the radiation-emitting components 410 and the underlying circuitry 402 from contaminants.

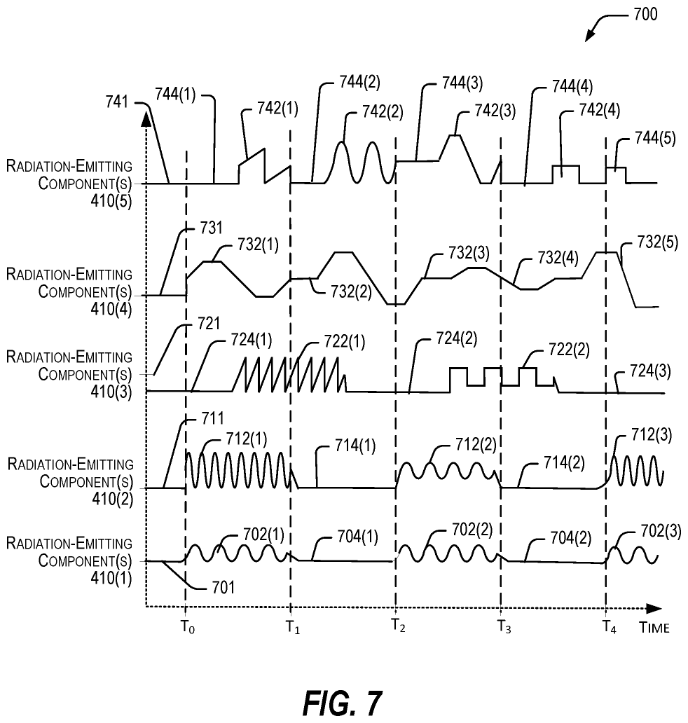

FIG. 7 depicts a graph 700 of electrical signals 701, 711, 721, 731, and 741 configured to drive selected radiation-emitting components 410 of a therapy module 102 modular therapy system 101 and having time-varying signal shapes, signal amplitudes, signal frequencies, and relative timing, in accordance with certain embodiments of the present disclosure. In this example, the time-varying signals are shown for multiple radiation-emitting components 410, which may be part of a single array 110 or multiple arrays 110. During the active periods of the arrays 110, the amplitude of the voltage signal may be greater than zero volts (i.e., above a ground signal), and the amplitude may vary between voltage levels from zero volts to one or more voltage levels that are greater than zero, as shown.

In this example, the signals 701, 711, 721, 731, and 741 may be provided to a single radiation-emitting component 410 or a subset of radiation-emitting components 410 of an array 110. The radiation-emitting component 410(1) may receive a signal 701 including a first periodic sinusoidal portion 702(1) from time T₀ to time T₁, a second periodic sinusoidal portion at 702(2) from time T₂ to time T₃, and a third periodic sinusoidal portion 702(3) from time T₄ to another time. In this example, the periodic sinusoidal portion 702 has four periods within each time window. The signal is constant (zero or turned off) at 704(1) from time T₁ to time T₂ and at 704(2) from time T₃ to T₄. In this example, the periodic sinusoidal portion 702 has a constant frequency and the common peak amplitude during the active periods.

The radiation-emitting component 410(2) may receive a second signal 711 having a first periodic sinusoidal portion at 712(1) having a first frequency from time T₀ to time T₁, a second periodic sinusoidal portion 712(2) having a second frequency from time T₂ to time T₃, and a third periodic sinusoidal portion 712(3) having the first frequency from time T₄ to another time. In this example, the periodic sinusoidal portions 712(1) and 712(3) have eight periods within each time window, and the periodic sinusoidal portion 712(2) has four periods within the time window. The signal 711 is constant (or turned off) at 714(1) from time T₁ to time T₂ and at 714(2) from time T₃ to T₄. In this example, the periodic sinusoidal portions 712 have the same peak amplitude during the active periods.

The radiation-emitting component 410(3) may receive a signal 721 having a periodic sawtooth portion 722(1) having a first frequency from a midpoint of a first time window (time T₀ to time T₁) to a midpoint of the second time window (time T₁ to time T₂). It is important to note that the timing of the beginning of the periodic sawtooth pattern is shifted to a midpoint of the first time window. The periodic sawtooth portion at 722(1) has a first frequency and is active for eight periods. From the beginning T₀ of the first time window until the periodic sawtooth portion 722(1) begins, the signal 721 is constant or turned off at 724(1). From a midpoint of a second time window (time T₁ to time T₂) until a midpoint of a third time window (time T₂ to time T₃), the signal 721 is turned off at 724(2). Beginning at the midpoint of the third time window until a midpoint of a fourth time window (from time T₃ to time T₄), the signal 721 includes a square wave portion at 722(2). From the midpoint of the fourth time window on, the signal 721 is constant (or turned off) at 724(3). In this example, the peak amplitude of the square wave 722(2) is less than the peak amplitude of the periodic sawtooth portion 722(1).

The radiation-emitting component 410(4) may receive a signal 731 including a periodic ramp portion having a first portion at 732(1) from time T₀ to time T₁, a second portion at 732(2) from time T₁ to time T₂, a third portion at 732(3) from time T₂ to time T₃, a fourth portion at 732(4) from time T₃ to time T₄, and a fifth portion at 732(5) from time T₄ on. In this example, the ramp portions 732 of the signal 731 has a constant frequency and a time-varying amplitude.

The radiation-emitting component 410(5) may receive a signal 741 including a first portion that is constant (or turned off) at 744(1) from time T₀ to a midpoint of the first time window. The signal 741 includes a periodic sawtooth portion 742(1) beginning at the midpoint of the first time window. The periodic sawtooth portion 742(1) may be applied for one period, ending at time T₁. In this example, the periodic sawtooth portion 742(1) is active for half the time window. The signal 741 may be constant (or turned off) at 744(2) from time T₁ until a midpoint of the second time window. The signal 741 may include a periodic sinusoidal portion 742(2) for two periods beginning at the midpoint of the second time window and ending at time T₂. The signal 741 may be constant (turned off) at 744(3) from time T₂ until a midpoint of the third time window and then the signal 741 may include a periodic ramp portion 742(3) for one period ending at time T₃. The signal 741 may be constant (turned off) at 744(4) from time T₃ until a midpoint of the fourth time window and then the signal 741 may a periodic square wave portion 742(4) for one period ending at time T₄. The signal 741 may be constant (turned off) at 744(5) from time T₄ on. In this example, the waveform types, the frequencies, and the amplitudes vary over time.

The therapy module 102 may modulate each radiation-emitting component 410 with different time-varying signals. The signals may vary in terms of on-off timing, frequency, amplitude, waveform type, duration, other parameters, or any combination thereof. In some implementations, according to some protocols, or in response to manual inputs, the therapy module 102 may modulate subsets of the radiation-emitting components 410 with different time-varying signals. In some implementations, in response to sensor signals, the MCU 106 of the therapy module 102 may selectively modulate one or more of the radiation-emitting components 410 to provide a selected therapeutic effect, such as pain mitigation, enhanced blood flow, or other effects.

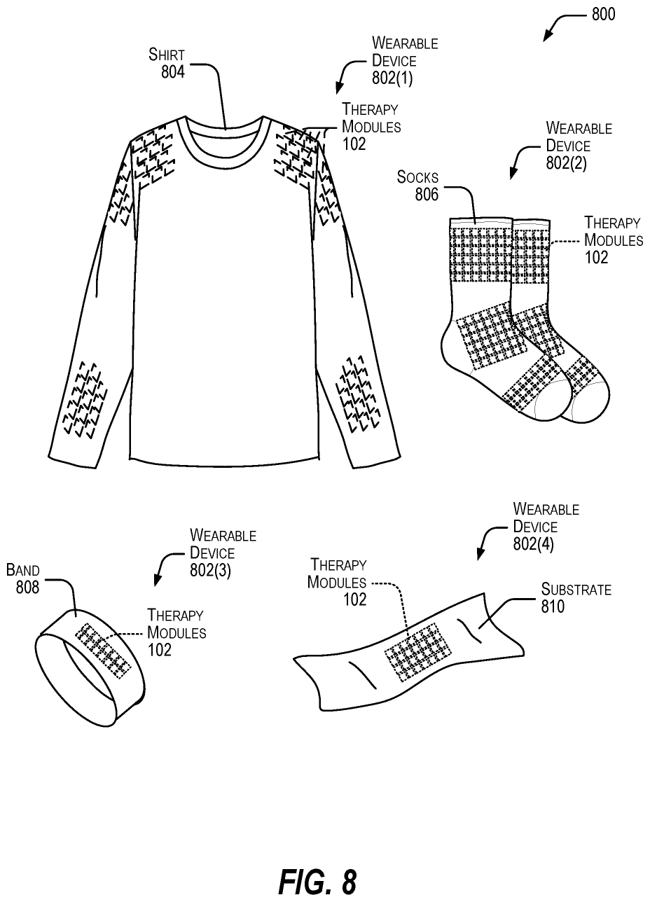

FIG. 8 depicts a system 800 including wearable devices 802 that may include therapy modules 102 integrated with or coupled to the wearable element and configurable to provide a selected therapeutic effect, in accordance with certain embodiments of the present disclosure. The wearable device 802(1) may include a shirt 804 including one or more arrays of therapy modules 102. In this example, the therapy modules 102 may be integrated into or coupled to the shirt 804 and arranged such that the radiation emitting components 410 are directed toward the skin of the patient.

The shirt 804 may include clusters of therapy modules 102 in various locations and may operate as a vehicle for securing the therapy modules 102 on the patient as he or she moves. In this non-limiting example, the therapy modules 102 are arranged about the shoulders and forearms of the shirt to provide treatment to the corresponding areas of the patient's skin. The shirt 804 may include therapy modules 102 at other locations, such as the chest, abdominal, lower back, upper back, elbow, wrist, and so on. In some implementations, the user may couple the therapy modules 102 to selected locations on the shirt to correspond to selected treatment locations. In an example, the therapy modules 102 may clip, pin, grip, or otherwise attach to the fabric of the shirt to secure the therapy modules 102 at one or more selected locations.

In some implementations, the shirt 804 may include a plurality of pockets or holders or attachment mechanisms that may be configured to secure the therapy modules 102 on the inside of the shirt at selected locations. In such an example, the user may selectively insert the therapy modules 102 into selected ones of the pockets, holders, or attachment mechanisms within the shirt 804 to position and secure the therapy modules 102 at selected locations to provide a selected treatment. Other implementations are also possible.

The wearable devices 802 may include socks 806, which may secure a plurality of therapy modules 102. The socks 806 may be worn by the patient, and the therapy modules 102 may be controlled to provide a selected therapeutic effect to one or more of the patients's calves, ankles, or feet.

The wearable devices 802 may include a band 808, such as an elastic headband, an elastic wristband, or an elastic band that may be worn on the patient's arm, calf, or leg. One or more therapy modules 102 may be integrated with or coupled to the band 808 and may be configured to provide a selected therapeutic effect to the area on which the band 808 is worn.

The wearable devices 802 may include a substrate 810, such as a towel, a blanket, a cloth, a wrap, or other type of flexible substrate. One or more therapy modules 102 may be coupled to or integrated with the substrate 810 and may be controlled to provide a selected therapeutic effect to an area covered by the substrate 810. In some implementations, the substrate 810 may include one or more adhesive areas adapted to couple the substrate 810 to the skin of the patient 902 to secure the therapy modules 102 relative to a treatment area.

The wearable devices 802 are depicted for illustrative purposes only and are not intended to be limiting. Specifically, the wearable devices 802 are not limited to those shown. Instead, the therapy modules 102 may be adapted to couple to any wearable article of clothing, such as a robe, a hat, pants, gloves, other clothing articles, bandages, other types of wraps, or any combination thereof.

In some implementations, the therapy modules 102 may be coupled to or integrated with a bandage that may be applied directly to a wound of a patient. The therapy modules 102 may be controlled to provide a selected therapeutic effect to the wound, such as killing bacteria, killing germs, enhancing blood flow to the injured area, encouraging skin cell production and healing, and so on.

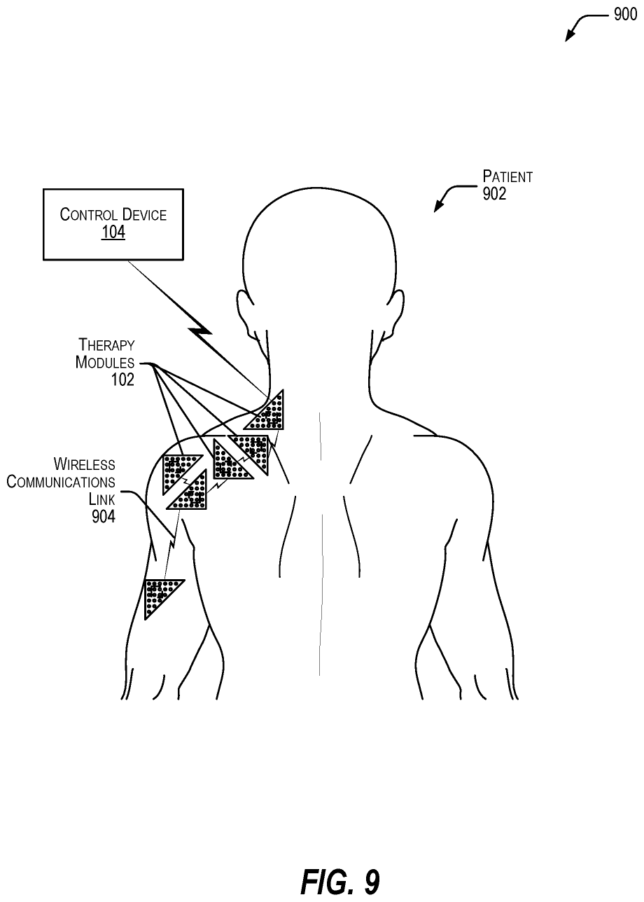

As mentioned above, the therapy modules 102 may applied directly to the patient's skin using an adhesive layer, which may be transparent to the radiated energy and which may (or may not) include medication. In some implementations, the therapy modules 102 may be applied to selected locations on the patient's body and may communicate via radio frequency communication links. An example of a therapy system 101 implemented as a plurality of therapy modules 102 applied directly to the skin of the patient is described below with respect to FIG. 9.

FIG. 9 depicts a system 900 including a plurality of therapy modules 102 applied directly to a patient's skin to form a distributed therapy system 101 configurable to provide a selected therapeutic effect, in accordance with certain embodiments of the present disclosure. In this example, an upper back of a patient 902 is depicted and six therapy modules 102 area applied directly to the patient 902 along the back of his or her shoulder and on the tricep muscle of the patient 902. The therapy modules 102 may include the transparent layer 632 (as shown and discussed in FIG. 6C), which may include an adhesive configured to releasably adhere the therapy module 102 to the skin of the patient 902. Other therapy modules 102 may also be applied to the patient 902 in other areas (not shown). The therapy modules 102 may be communicatively coupled to a control device 104 and to one another via wireless communication links 904 to provide a therapy system 101 that may be controlled to provide a selected therapeutic effect.

In some implementations, the adhesive layer may be transparent to the radiation emitted by the therapy modules 102. In some implementations, in addition to securing the therapy modules 102 to the skin of the patient 902, the adhesive layer may also provide a delivery mechanism for medication. In some instances, one or more therapy modules 102 may be applied directly to a wound, and the adhesive layer may operate to seal the afflicted area from the surrounding environment, deliver selected medication to the afflicted area, and enable application of emitted radiation to the afflicted area via the therapy modules 102.

In an example, the therapy modules 102 may be distributed on a wounded patient such that some of the therapy modules 102 are applied to the wound while others of the therapy modules 102 are applied to other areas. The therapy modules 102 that are applied to the wound may be controlled to kill bacteria and prevent infection, to reduce pain, to enhance blood flow, to promote cell proliferation (fibrous tissue cells, epidermal cells, and endothelial cells), or to facilitate other therapeutic effects to facilitate healing of the wound.

In another example, the therapy modules 102 may be distributed on the patient to facilitate one or more selected therapeutic effects. For an arm injury, for example, the therapy modules 102 may be configured to deliver a heating effect, to enhance blood flow through the injured area and to other adjacent areas of the patient's body, to decrease inflammation, to mitigate pain, to provide other therapeutic effects, or any combination thereof.

In some implementations, spacing between the therapy modules 102 may allow the patient 902 to move freely (without restriction from the therapy modules 102). The therapy modules 102 may communicate with one another and optionally with the control device 104 via the wireless communication links 904 to coordinate timing of operations. Other implementations are also possible.

The adhesive layer may allow the therapy modules 102 to adhere to the patient 904 while the patient 904 exercises or performs physical therapy-related movements, thereby enhancing the physical therapy process. Additionally, the adhesive layer may allow the therapy modules 102 to be applied to areas on the patient 902 that might otherwise be difficult to reach. Further, the adhesive layer that couples the therapy module 102 to the patient 902 may also serve as a vehicle for delivering medicine to the patient's skin or to a wound.

While the therapy modules 102 are depicted as right triangular shaped modules, it should be understood that the therapy modules 102 may be formed from any number of selected shapes. As previously discussed, two or more of the therapy modules 102 may be coupled together and others of the therapy modules 102 may be deployed separately, forming one or more therapy systems 101 that may be controlled by the control device 104 to provide a selected therapeutic effect.

In this example, the therapy modules 102 are deployed on the upper back area and on the triceps of the patient 902. However, depending on the treatment to be employed, the therapy modules 102 may be deployed at other locations on the body of the patient 902. The adhesive layer may enable selective deployment to any location on the patient 902.

While the therapy modules 102 are depicted as communicating via wireless communications links, in some implementations, one or more of the communication links may be wired and others may be wireless. In addition to communications, wired connections may also enable power sharing between batteries of connected therapy modules 102.

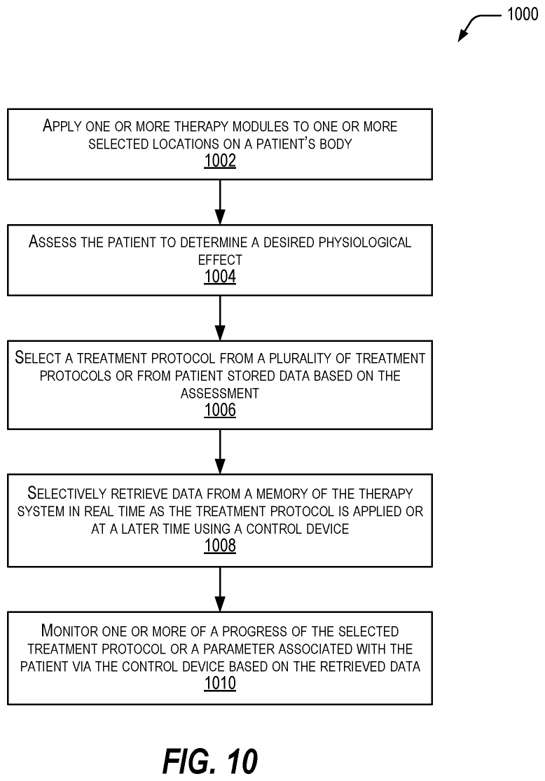

FIG. 10 depicts a flow diagram of a method 1000 of monitoring one or more parameters associated with a patient, in accordance with certain embodiments of the present disclosure. At 1002, the method 1000 may include applying one or more therapy modules 102 to one or more selected locations on a patient's body. As discussed with respect to FIG. 9, each of the one or more therapy modules 102 may be secured at the one or more selected locations by a transparent layer 632, which may operate as an adhesive and which may include topical or transdermal medicine. The selected locations may include treatment locations the body of the patient 902, such as his or her shoulder, arm, elbow, wrist, hand, neck, chest, back, buttocks, hip, thigh, groin, knee, shin, ankle, other area, or any combination thereof. An operator, such as a therapist, may apply the one or more therapy modules 102 to the selected locations.

At 1004, the method 1000 may include assessing the patient to determine a desired physiological effect. In some implementations, the patient may be assessed by a treatment professional. In other implementations, the patient may be assessed by a machine learning algorithm, an artificial intelligence engine, a neural network, a complex algorithm, or a series of filters based on data into a computing device (such as the control device 104 or a computing device 144) or based on data determined automatically by the system via optical analysis or other analytics.

At 1006, the method 1000 may include selecting a treatment protocol 122 from a plurality of treatment protocols 122 or from patient stored data based on the assessment. In some implementations, the treatment protocols 122 or the patient stored data may be stored in a memory 214 of the control device 104 or in a memory 112 of each therapy module 102. The control device 104 may be a computing device that presents a graphical interface including user-selectable control options accessible by an operator to select one of the treatment protocols 122. The treatment protocols 122 may define one or more of the waveform type, the frequency, the amplitude, the duration, and the timing of signals provided to one or more radiation-emitting components of an array 110, controlling the frequency, intensity, timing, and duration of electromagnetic radiation applied to the patient's body. Based on the selected protocol 122, the control device 104 may send one or more signals to the therapy system 101, to individual therapy modules 102 of the system 101, to selected subsets of radiation-emitting components 410 of one or more of the arrays 110 of the therapy modules 102, to selected radiation-emitting components 410, or any combination thereof.

At 1008, the method 1000 may include selectively retrieving data from a memory 112 of the therapy module 102 in real time as the treatment protocol is applied or at a later time using a control device 104. In some implementations, the therapy module 102 may store sensor data and other data in the memory 112 during operation and may provide the sensor data to a control device 104 or an analytics system 146 via the control device 104 at a later time. In other implementations, the therapy module may store the sensor data and other data in the memory 112 and communicate the sensor data to the control device 104. In still other implementations, the microcontroller unit 106 of the therapy module 102 may selectively process the sensor data.

At 1010, the method 1000 may include monitoring one or more of a progress of the selected treatment protocol 122 or a parameter associated with the patient via the control device 104. The control device 104 or an MCU 106 of the therapy module 102 may be configured to monitor data from one or more sensors 128 or the timing of the execution of the selected protocol 122. In other implementations, the control device 104 may communicate the sensor data or other data to an analytics system 146, which may monitor the progress.

In some implementations, one or more of the control device 104 or each therapy module 102 may be configured to monitor sensor signals from one or more sensors 128 to determine the patient's response to the selected protocol 122 over time. In some implementations, one or more of the control device 104 or the therapy module 102 may adjust one or more signals based on the sensor signals.

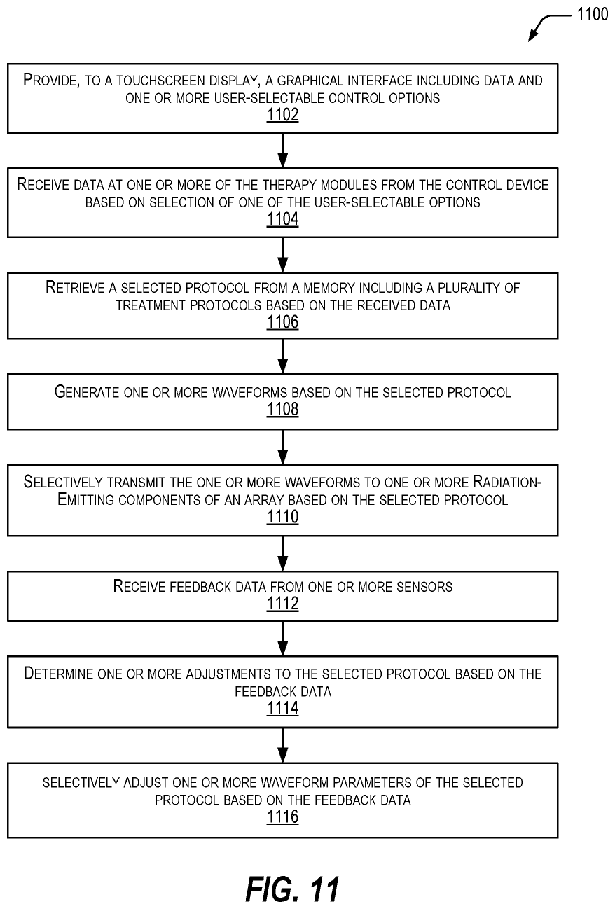

FIG. 11 depicts a flow diagram of a method 1100 of selectively adjusting one or more parameters of a selected treatment mode, in accordance with certain embodiments of the present disclosure. The system including the therapy system 101 coupled to the control device 104 may enable manual selection of a protocol 122 and optionally manual (via a graphical interface) or automatic adjustment of the selected protocol 122 via automated decision-making by one or more of the MCU 106 of one of the therapy modules or by a processor 210 of the control device 104.

At 1102, the method 1100 may include providing, to a touchscreen display, a graphical interface including data and one or more user-selectable control options. The data may include images, text, instructions, and other information. The user-selectable control options may include buttons, tabs, clickable links, buttons textboxes, radio buttons, pull-down menus, text fields, or any combination thereof. The user may interact with the control device 104 to specify a selected protocol 122 and other data. The control device 104 may receive the input data via an I/O interface 216. In some implementations, one of the user-selectable control options may include a first option for the user to select one of a plurality of treatment protocols 122, a second option to edit one or more of the user-selectable control options, and a third option to specify manual or automatic control. The control device 104 may send data related to the selection of the user-selectable control options to the therapy system 101 (to one or more therapy modules 102).

At 1104, the method 1100 may include receiving data at one or more of the therapy modules 102 from the control device 104 based on selection of one of the user-selectable options. The received data may specify a selected protocol 122 and other parameters.

At 1106, the method 1100 may include retrieving a selected protocol 122 from a memory 112 including a plurality of treatment protocols 122 based on the received input data. The selected protocol 122 may specify timing, intensity, wavelength, and duration of electromagnetic radiation to be applied as a treatment to selected areas of the patient's body.

At 1108, the method 1100 may include generating one or more waveforms based on the selected protocol 122. The waveforms may have selected waveform shapes (sinusoidal, square, sawtooth, ramp, etc.), selected amplitudes, selected wavelengths, selected timing, selected duration, or any combination thereof. The waveforms may be periodic or aperiodic.

At 1110, the method 1100 may include selectively transmitting one or more waveforms to one or more radiation-emitting components 410 of an array 110 based on the selected protocol 122. The waveforms may be provided to the array 110 or to selected radiation-emitting components 410 of the array 110.

At 1112, the method 1100 may include receiving feedback data from one or more sensors 128. The feedback data may be received at an MCU 106 of the therapy module 102. The MCU 106 may process the feedback data (sensor data) to determine adjustments. Alternatively, the MCU 106 may send the feedback data to a control device 104 and may receive data from the control device 104 in response to the feedback data.

At 1114, the method 1100 may include determining one or more adjustments to the selected protocol 122 based on the feedback data. The MCU 106 may determine the one or more adjustments based on the feedback data. For example, if the MCU 106 determines from the feedback data that the temperature of the patient's skin is higher than a threshold temperature, the MCU 106 may deactivate one or more of the radiation-emitting components 410. In another example, if the MCU 106 determines from the feedback data indicates that the sensed blood flow parameters are within a target range, the MCU 106 may cause the waveform generator 108 to adjust one or more of the amplitude, duration, frequency, timing, or other parameters of the waveform to maintain the blood flow while reducing electromagnetic radiation applied to the patient. In still another example, if the MCU 106 determines that an amount of time of exposure of the patient to the electromagnetic radiation exceeds a time threshold, the MCU 106 may deactivate the array 110. Other parameters may be evaluated by the MCU 106 to determine other adjustments.

At 1116, the method 1100 may include selectively adjusting one or more waveform parameters of the selected protocol based on the feedback data. In an example, the MCU 106 may cause the waveform generator 108 to adjust one or more of an amplitude or a duty cycle for driving a selected one or more radiation-emitting components 410. Other implementations are also possible.

In the method 1100, the MCU 106 may determine the adjustments from the selected protocol 122. In other implementations, the MCU 106 may send the feedback data to the control device 104 and the control device 104 may determine the adjustments and send them to one or more of the therapy modules 102.

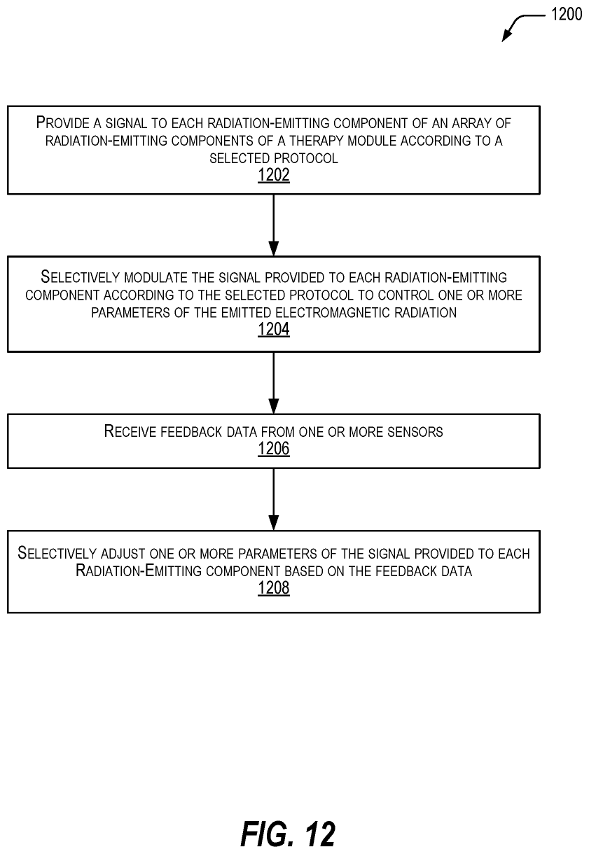

FIG. 12 depicts a flow diagram of a method 1200 of selectively adjusting one or more parameters of signals provided to radiation-emitting components 410 of the therapy module 102, in accordance with certain embodiments of the present disclosure. At 1202, the method 1200 may include providing a signal to each radiation-emitting component 410 of an array 110 of radiation-emitting components of a therapy module 102 according to a selected protocol 122. The MCU 106 may cause the waveform generator 108 to produce one or more signals based on the selected protocol 122.

At 1204, the method 1200 may include selectively modulating the signal provided to each radiation-emitting component 410 according to the selected protocol 122 to control one or more parameters of the emitted electromagnetic radiation. The MCU 106 may modulate the signal provided to each radiation-emitting component 410 by controlling the waveform generator 108 to produce modulated signals.

At 1206, the method 1200 may include receiving feedback data from one or more sensors 128. The feedback data may be received by the MCU 106 from integrated sensors, from external sensors, or any combination thereof.

At 1208, the method 1200 may include selectively adjusting one or more parameters of the signal provided to each radiation-emitting component based on the feedback data. In an example, the MCU 106 may determine adjustments to the selected protocol 122 based on the feedback data. In other examples, the MCU 106 may send the data to a control device 104, which may determine the adjustments.

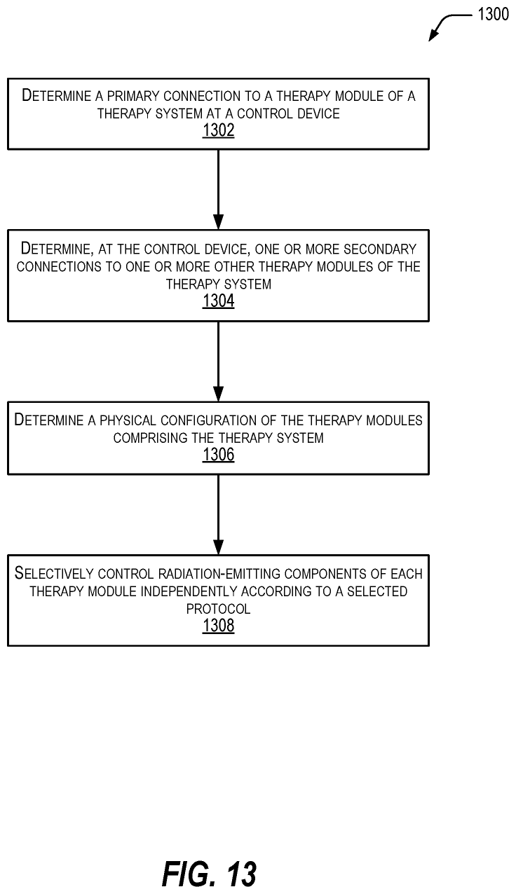

FIG. 13 depicts a flow diagram of a method 1300 of selectively controlling radiation-emitting components 410 of each therapy module 102, independently, in accordance with certain embodiments of the present disclosure. At 1302, the method 1300 may include determining a primary connection to a therapy module 102(1) of a therapy system 101 at a control device 1302. In some implementations, the primary connection may be determined based on a first therapy module 102(1) to communicatively couple to the control device 104. In other implementations, the primary connection may be determined based on a physical connection between the control device 104 and the first therapy module 102(1).

At 1304, the method 1300 may include determining, at the control device 104, one or more secondary connections to one or more other therapy modules 102 of the therapy system 101. In some implementations, the control device 104 may determine the secondary connections during a calibration process during which the control device 104 receives identifier information and connection information from each of the therapy modules 102. In some implementations, the control device 104 may be configured to determine a communication path from the control device 104 to each of the therapy modules 102 through the primary therapy module 102(1).

At 1306, the method 1300 may include determining a physical configuration of the therapy modules 102 comprising the therapy system 101. In an example, the control device 104 may include the identifiers and connection data for each of the therapy modules 102. The control device 104 may assemble a virtual map of the configuration of therapy modules 102 that form the therapy system 101.

At 1308, the method 1300 may include selectively controlling radiation-emitting components 410 of each therapy module 102 independently according to a selected protocol 122. The control device 104 may send control signals to each of the one or more therapy modules 102, which may cause each therapy module 102 to selectively control one or more radiation-emitting components 104.

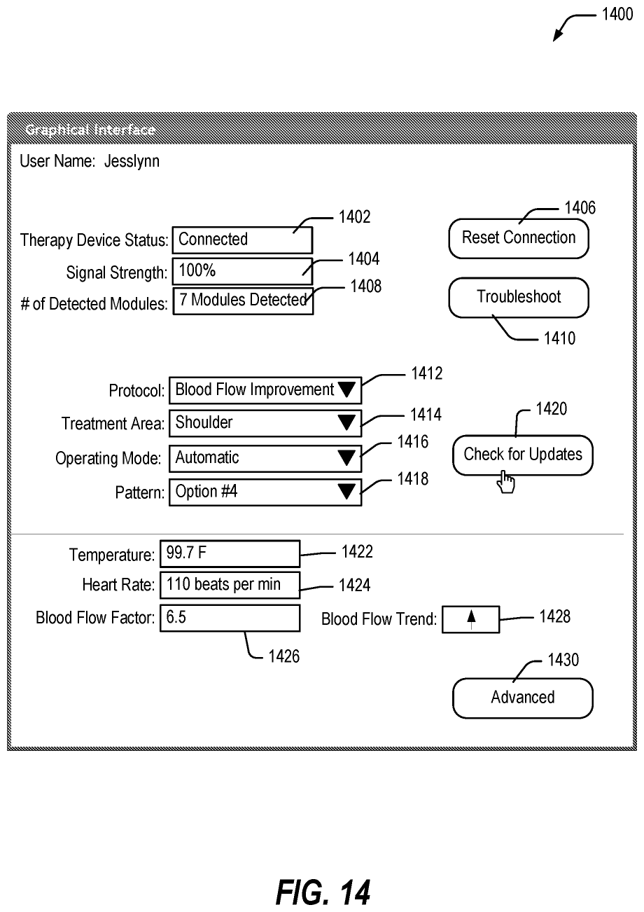

In some implementations, the control interface 104 may provide a graphical interface to a display that may include text and user-selectable control options accessible by an operator to selectively control operation of the therapy system 101. An illustrative non-limiting example of a graphical interface is described below with respect to FIG. 14.

FIG. 14 depicts a graphical interface 1400 including data and user-selectable control options, in accordance with certain embodiments of the present disclosure. The graphical interface 1400 is depicted for illustrative, non-limiting purposes only and is not intended to be limiting or to represent an exhaustive list of the parameters, settings, or control options available to a user. Instead, the graphical interface 1400 is presented to show that the user may interact with a graphical interface to select a protocol, to specify treatment areas, to specify operating modes, to configure patterns or settings, and optionally to monitor various parameters associated with the patient 902 (as determined from data received from the sensors 128). Depending on the implementations and depending on settings configured by the user, other interfaces, other selectable control options, and other data may be presented.

The graphical interface 1400 may include a therapy device status indicator 1402, which in this example indicates that the control device 104 is connected. The graphical interface 1400 may include a signal strength indicator 1404, which in this example indicates that the signal strength is 100%. The graphical interface 1400 also includes data indicative of a number of detected therapy modules 1408, which in this example indicates that seven therapy modules are detected. The graphical interface 1400 may include a “Reset Connection” button 1406, which may be accessed by an operator to trigger the control device 104 to reset the connection. The graphical interface 1400 may include a “Troubleshoot” button 1410, which may be accessed by the operator to determine and resolve connection issues. In some implementations, upon selection of the Troubleshoot button 1410, the graphical interface 1400 may display instructions for the operator to assist in a troubleshooting operation.

The graphical interface 1412 may include a first control option 1412 to select a protocol 122 from a plurality of protocols. In this example, the operator has selected a “Blood Flow Improvement” protocol using the first control option 1412. Other protocols 122 may include pain remediation, blood flow enhancement, tissue treatment, surface heating, surface treatment, other treatments, and so on. The different protocols may use different amplitudes, different frequencies, different durations, different timing, and other differences.

The graphical interface 1400 may include a second control option 1414 to select a treatment area from a plurality of treatment areas on the patient's body. In this example, the operator has selected “Shoulder” using the second control option 1414. In some implementations, the plurality of treatment areas may include the shoulder, arm, elbow, wrist, hand, neck, chest, back, buttocks, hip, thigh, groin, knee, shin, ankle, another treatment area, or any combination thereof. The control option 1414 may be accessed by the operator to select multiple treatment areas.

The graphical interface 1400 may include a third control option 1416 to select an operating mode from a plurality of operating modes. In this example, the operator has selected “Automatic,” which may allow the MCU 106 of each therapy module 102 or the processor 210 of the control device 104 to automatically adjust the waveforms provided to selected ones of a plurality of radiation-emitting components 410 of an array 110 based on feedback signals. The third control option 1416 may allow the operator to select between automatic or manual operating modes. In the manual operating mode, sensor feedback may be provided to the graphical display 1400 with text suggestions for adjustments and user-selectable control options accessible by the operator to manually adjust one or more settings, one or more waveforms, one or more protocols, or any combination thereof.

The graphical interface 1400 may include a fourth user-selectable control option 1418 to select a pattern from a plurality of electromagnetic radiation patterns. In this example, the operator has selected “Option #4”, which may cause one or more of the therapy modules 102 to control the waveform generator 108 to control the radiation-emitting components 410 according to the selected pattern. The fourth user-selectable control option 1418 may include a list of pattern options, which may include custom patterns or other patterns.

The graphical interface 1400 may include a “Check for Updates” button 1420, which may be accessed by the operator to cause the control device 104 to determine whether there are updates for the control device 104. The updates may include new operating modes, new patterns, new protocols, other options, or any combination thereof. In some implementations, the updates may include a new graphical interface with other user-selectable control options.

In some implementations, the graphical interface 1400 may display sensor data. The sensor data may include temperature data 1422. In this example, the temperature data 1422 may be higher than 98.6 degrees Fahrenheit because electromagnetic radiation emitted by the therapy system 101 may cause the surface temperature of the patient's skin to increase.

The sensor data may include heart rate data 1424. In this example, the heart rate data 1424 indicates “110 beats per minute”, which may be indicative of elevated heart rate due to pain, for example.

In this example, the selected treatment protocol is for improved blood flow, and the sensors 128 may monitor capillary blood flow, blood pressure, pulse rate, and other parameters associated with the patient. In this example, the graphical interface 1400 includes a blood flow factor 1426, which may be a number indicative of the quality of blood flow in the patient's shoulder or arm based on the sensor data. In this example, the blood flow factor 1426 indicates a factor of 6.5 on a scale from 1 to 10, where a factor of 10 is indicative of excellent oxygenation of the tissue while a factor of 1 may be indicative of a blockage or impingement requiring further treatment. In this example, the graphical interface 1400 may also include a blood flow trend indicator 1428, which is trending up in this example. The blood flow factor 1426 and the blood flow trend indicator 1428 may assist the operator to determine improvements over time, and changes in these may cause the operator, the control device 104, or the MCUs 106 of the various therapy modules 102 to adjust the protocol 122. The graphical interface 1400 may include an “Advanced” button 1430, which may be accessed by the operator to view additional data and features.

It should be appreciated that the graphical interface 1400 depicted in FIG. 14 is an illustrative, non-limiting example. The contents, the types of control options, and other features presented in the graphical interface 1400 may be changed without departing from the spirit and scope of this disclosure.

In conjunction with the systems, methods, devices, and interfaces described above with respect to FIGS. 1-14, a system is disclosed that may include a control device 104 configured to communicate with and control the operation of one or more therapy systems 101, each which may be formed from one or more therapy modules 102. Each therapy module 102 may include a plurality of radiation-emitting components 410 in an array 110. The therapy module 102 may control each of the radiation-emitting components 410 individually, in subsets, or across the entire array.

The therapy module 102 may include a waveform generator 108 configured to generate a selected waveform (sine wave, square wave, sawtooth wave, ramp wave, etc.) having a selected amplitude, a selected frequency, a selected duration, and selected timing to control emission of electromagnetic radiation according to a selected protocol 122. In some implementations, the waveform generator 108 may vary one or more parameters of the selected waveform over time for each of the radiation-emitting components 410.

In the above-discussion, the array 110 is described as being comprised of radiation-emitting components 410. However, the array 110 may be comprised of other types of components configured to emit human-safe electromagnetic radiation toward the patient's skin to provide a selected therapeutic effect.

The one or more therapy modules 102 may be configured to communicate with a control device 104. The one or more therapy modules 102 may receive control signals from the control device 104, perform a selected protocol based on the control signals, determine sensor data associated with the user, and provide the sensor data to the control device 104. The control device 104 may include a smartwatch, a smartphone, a tablet computing device, another computing device or electronic device, or any combination thereof.

Although the present invention has been described with reference to preferred embodiments, workers skilled in the art will recognize that changes may be made in form and detail without departing from the scope of the invention.

What is claimed is:

1. A system comprising:
a therapy device configured to couple to a patient, the therapy device comprising one or more therapy modules, each therapy module comprises:
an array of radiation-emitting components, each emitting component configured to emit electromagnetic radiation in response to an electrical signal; and
a waveform generator coupled to the array of radiation-emitting components and configured to generate one or more time-varying waveforms to drive the radiation-emitting components; and
a processor coupled to the waveform generator and to the array of radiation-emitting components, the processor configured to:
select a protocol of a plurality of protocols, each protocol defining one or more parameters, the one or more parameters including one or more of a waveform shape, an amplitude, a frequency, a timing parameter, and a duration parameter; and
send a signal to the waveform generator to generate the one or more time-varying waveforms according to the selected protocol.

2. The system of claim 1, wherein:
the plurality of protocols includes one or more of a sinusoidal waveform, a square waveform, a sawtooth waveform, a triangle waveform, or a ramp waveform; and
the one or more time-varying waveforms vary in terms of one or more of the waveform shape, the amplitude, the frequency, the timing parameter, or the duration parameter.

3. The system of claim 1, wherein each emitting component may be configured to emit light having a selected wavelength and a selected amplitude within a range of the electromagnetic spectrum including visible light, infrared light, and ultraviolet light.

4. The system of claim 1, further comprising a plurality of sensors, each sensor configured to generate an electrical signal indicative of one or more of contaminants on the patient or physiological parameters of the patient.

5. The system of claim 4, wherein the plurality of sensors comprises one or more of resistive sensors, capacitive sensors, optical sensors, radiant sensors, biosensors, chemical sensors, or pressure sensors.

6. The system of claim 4, wherein the plurality of sensors includes one or more optical sensors configured to determine birefringence of one or more of water or tissue of a patient.

7. The system of claim 4, wherein the physiological parameters include one or more of blood flow, blood pressure, birefringence, temperature, blood sugar, chemical contents of sweat, or data related to one of skin, soft tissue, muscle, bone, or fascia of the patient.

8. The system of claim 4, wherein the processor is configured to:
receive the electrical signals from the one or more sensors indicative of the one or more of contaminants or the physiological parameters; and
send a signal to the waveform generator to selectively adjust at least one of the one or more time-varying waveforms based on the received electrical signals from the one or more sensors.

9. The system of claim 1, wherein each therapy module includes a device interface; and
wherein the system further comprises:
a control device configured to establish a communications link with the device interface and configured to communicate data and control instructions to the therapy device via the communications link; and
wherein the control device comprises one of a smartwatch, a smartphone, a laptop computer, a tablet computer, or a control system of a patient transport vehicle.

10. The system of claim 1, wherein the waveform generator is configured to selectively control each emitting component independently.

11. The system of claim 1, further comprising:
a transparent layer between the array of radiation-emitting components and the patient and configured to secure the therapy device to the patient;
wherein the emitted electromagnetic radiation passes through the transparent layer to the patient; and
wherein the transparent layer comprises one or more of an adhesive or a medicinal coating.

12. A system comprising:
a control device including an input interface to receive input data and a communications interface;
one or more therapy devices configured to couple to a patient, each therapy device including one or more therapy modules, at least one of the one or more therapy modules including a device interface configured to determine a communications link between the device interface and the communications interface of the control device, each therapy module including:
an input/output (I/O) interface configured to communicatively couple to an adjacent therapy module;
an array of radiation-emitting components configured to emit electromagnetic radiation toward the patient;
a waveform generator coupled to the array and configured to generate one or more time-varying waveforms in response to a control signal; and
a processor configured to:
receive data related to the input data from the device interface;
determine a selected protocol from a plurality of protocols based on the received data; and
send the control signal to the waveform generator based on the selected protocol to treat one or more treatment areas on the patient according to the selected protocol.

13. The system of claim 12, wherein:
the plurality of protocols includes one or more of a sinusoidal waveform, a square waveform, a sawtooth waveform, a triangle waveform, or a ramp waveform; and
the one or more time-varying waveforms vary in terms of one or more of the waveform shape, the amplitude, the wavelength, the timing parameter, or the duration parameter.

14. The system of claim 12, wherein each emitting component may be configured to emit light having a selected wavelength and a selected amplitude within a range of the electromagnetic spectrum including visible light, infrared light, and ultraviolet light.

15. The system of claim 1, further comprising a plurality of sensors, each sensor configured to generate an electrical signal indicative of one or more of contaminants on the patient or physiological parameters of the patient.

16. The system of claim 15, wherein the processor is configured to:
receive the electrical signals from the one or more sensors indicative of the one or more of contaminants or the physiological parameters; and
send a signal to the waveform generator to selectively adjust at least one of the one or more time-varying waveforms based on the received electrical signals from the one or more sensors; and
wherein the waveform generator is configured to selectively control each emitting component independently.

17. The system of claim 15, wherein the plurality of sensors includes one or more optical sensors configured to determine birefringence of one or more of water or tissue of a patient.

18. The system of claim 12, further comprising:
a transparent layer between the array of radiation-emitting components and the patient and configured to secure each of the one or more therapy devices to the patient;
wherein the emitted electromagnetic radiation passes through the medicinal coating or the transparent layer to the patient; and
wherein the transparent layer comprises one or more of an adhesive or a medicinal coating.

19. A system comprising:
a plurality of therapy modules configured to couple to one or more treatment areas on a patient, each therapy module comprising:
a device interface configured to selectively establish a communications link to a control device;
one or more input/output (I/O) interfaces configured to communicatively couple the therapy module to one or more other therapy modules to form a therapy device having a selected size and shape;
an array of radiation-emitting components configured to emit electromagnetic radiation toward one of the one or more treatment areas;
a waveform generator coupled to the array and configured to generate one or more time-varying waveforms to drive individual radiation-emitting components, subsets of the radiation-emitting components, or all of the radiation-emitting components of the array; and
a processor configured to:
select a protocol from a plurality of protocols; and
control the waveform generator to generate the one or more time-varying waveforms based on the selected protocol.

20. The system of claim 19, further comprising the control device including a graphical interface through which an operator specifies the protocol from the plurality of protocols, the control device receives input data related to the specified protocol and communicates selection data to the processor of the therapy device through the communications link.
